(19) United States
(12) Patent Application Publication
(10) Pub. No.: US 2004/0192693 A1
(43) Pub. Date: Sep. 30, 2004

(54) Aminoalkyl-substituted aromatic bicyclic compounds, methods for their preparation and their use as pharmaceuticals

(75) Inventors: Lothar Schwink, Stadtallendorf (DE); Siegfried Stengelin, Eppstein (DE); Matthias Gossel, Hofheim (DE); Armin Walser, Tuscon, AZ (US); Gerard Rosse, Oro Valley, AZ (US)

Correspondence Address: HELLER EHRMAN WHITE & MCAULIFFE LLP, 1666 K STREET,NW, SUITE 300, WASHINGTON, DC, 20006, US

(73) Assignee: Aventis Pharma Deutschland GmbH, Frankfurt am Main (DE)

(21) Appl. No.: 10/820,706

(22) Filed: Apr. 9, 2004

Related U.S. Application Data
(62) Division of application No. 10/218,034, filed on Aug. 14, 2002.

(30) Foreign Application Priority Data
Aug. 17, 2001 (DE) 10139416.0

(51) Int. Cl. A61K 31/53; A61K 31/497; A61K 31/4439; C 07D 4 3/14

(52) U.S. Cl. 514/242; 514/252.06; 514/255.05; 514/338; 544/182; 544/238; 544/405; 546/268.4

(57) ABSTRACT

The present invention relates to aminoalkyl-substituted aromatic bicyclic compounds of formula I,

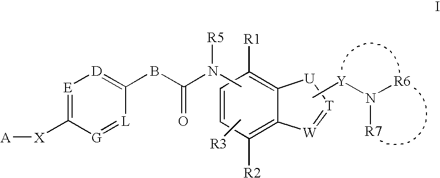

which are valuable pharmaceutically active compounds that are suitable, for example, for the treatment of obesity, type II diabetes, arteriosclerosis, high blood pressure, paresthesia, depression, anxiety, anxiety neuroses, schizophrenia, disorders associated with the circadian rhythm, and drug abuse, as well as normalizing lipid metabolism.

This application claims priority to German Patent Application 10139416.0, filed Aug. 17, 2001, which is hereby incorporated by reference, in its entirety. All references cited below, including patents, patent applications and scientific journals and books also are herein incorporated by reference in their entirety.

### FIELD OF THE INVENTION

The invention relates to aminoalkyl-substituted aromatic bicyclic compounds and to the physiologically acceptable salts and physiologically functional derivatives thereof.

### BACKGROUND OF THE INVENTION

Structurally similar nonaromatic bicyclic compounds with pharmacological action have already been described in the prior art (for example in WO 01/21577).

The present invention provides compounds which cause a reduction in weight in mammals and which are suitable for preventing and treating obesity and diabetes.

### SUMMARY OF THE INVENTION

The present invention relates to aminoalkyl-substituted aromatic bicyclic compounds of formula I,

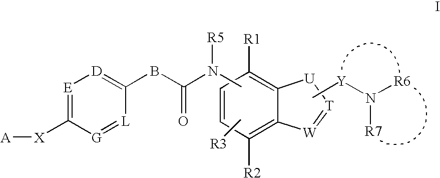

wherein A, X, D, E, G, L, B, R5, R1, R2, R3, W, U, T, Y, R6 and R7 have the meanings as indicated herein. The compounds of formula I are valuable pharmaceutically active compounds which are suitable, for example, for the treatment of obesity, type II diabetes, arteriosclerosis, high blood pressure, paresthesia, depression, anxiety, anxiety neuroses, schizophrenia, disorders associated with the circadian rhythm, and drug abuse, as well as normalizing lipid metabolism.

### DETAILED DESCRIPTION OF THE EMBODIMENTS

The invention therefore relates to compounds of formula I,

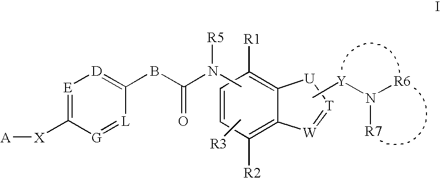

in which

A is (C₁-C₈)alkyl, (C₀-C₈)alkylenearyl, or a 3- to 12-membered mono- or bicyclic ring which may contain one or more heteroatoms selected from the group consisting of N, O and S and the 3- to 12-membered ring may carry further substituents, such as F, Cl, Br, NO₂, CF₃, OCF₃, CN, (C₁-C₆)alkyl, aryl, CON(R37)(R38), N(R39)(R40), OH, O—(C₁-C₆)alkyl, S—(C₁-C₆)alkyl, or NHCO(C₁-C₆)alkyl;

X is a bond, C(R8)(R9), C(OR10)(R11), O, N(R12), S, SO, SO₂, or CO; wherein R8, R9, R10, R11, R12 are, independently of one another, H, (C₁-C₆)alkyl;

D is N, or C(R41);

E is N, or C(R42);

G is N, or C(R43);

L is N, or C(R44);

R1, R2, R3, R41, R42, R43, R44 are, independently of one another, H, F, Cl, Br, J, OH, CF₃, NO₂, CN, OCF₃, O—(C₁-C₆)alkyl, (C₁-C₄)alkoxyalkyl, S—(C₁-C₆)alkyl, (C₁-C₆)alkyl, (C₂-C₆)alkenyl, (C₃-C₈)cycloalkyl, O—(C₃-C₈) cycloalkyl, (C_{3-C}₈)cycloalkenyl, O—(C₃-C₈)cycloalkenyl, (C₂-C₆)alkynyl, (C₀-C₈)alkylenearyl, —O—(C₀-C₈)alkylenearyl, S-aryl, N(R13)(R14), SO₂-CH₃, COOH, COO—(C₁-C₆)alkyl, CON(R15)(R16), N(R17)CO(R18), N(R19)SO₂(R20), CO(R21), or a 5- to 7-membered heterocycle having 1-4 heteroatoms;

R13, R14 are independently of one another H, (C₁-C₆)alkyl, or R13 and R14 together with the nitrogen atom to which they are bonded form a 5- to 6-membered ring, where, in the case of the 6-membered ring, a CH₂ group may be replaced by O or S;

R15, R16 are independently of one another H, (C₁-C₆)alkyl, or R15 and R16 together with the nitrogen atom to which they are bonded form a 5- to 6-membered ring, where, in the case of the 6-membered ring, a CH₂ group may be replaced by O or S;

R17, R19 are independently of one another H, or (C₁-C₆)alkyl;

R18, R20, R21 are independently of one another (C₁-C₆)alkyl, or aryl;

B is N(R24), or O;

R24 is H, or (C₁-C₆)alkyl;

R5 is H, or (C₁-C₆)alkyl;

W is N, or C(R25);

R25 is H, (C₁-C₆)alkyl, aryl, or a bond to Y;

T is N, or C(R26);

R26 is H, (C₁-C₆)alkyl, aryl, (C₀-C₈)alkylenearyl, or a bond to Y;

U is O, S, N(R27), —C(R30)═N—, or —N═C(R31)—;

wherein R27, R30, R31 are independently of one another H, (C₁-C₆)alkyl, a bond to Y;

Y is (C₁-C₈)alkylene, in which one or more carbons may be replaced by O, S, SO, SO₂, C(R32)(R33), CO, C(R34)(0R35) or N(R36);

R32, R33, R34, R35, R36 are independently of one another H, (C₁-C₆)alkyl, or aryl;

R6, R7 are independently of one another H, (C₁-C₆)alkyl, (C₃-C₇)cycloalkyl, or R6 and Y or R6 and R7 together with the nitrogen atom to which they are bonded form a 3- to 8-membered ring in which one or more carbons may be replaced by O, N or S and the 3- to 8-membered ring may carry further substituents, such as (C₁-C₆)alkyl, aryl, CON(R37)(R38), N(R39)(R40), OH, O—(C₁-C₆)alkyl or NHCO(C₁-C₆)alkyl;

R37, R38, R39, R40 are independently of one another H, or (C₁-C₆)alkyl;

and the physiologically acceptable salts thereof.

Preference is given to compounds of formula I, in which one or more radicals have the following meaning:

A is (C₂-C₇)alkyl, (C₀-C₃)alkylenearyl; or a 4- to 10-membered mono- or bicyclic ring which may contain one or more heteroatoms selected from the group consisting of N, O and S, and the 4- to 10-membered ring may carry further substituents, such as F, Cl, Br, NO₂, CF₃, (C₁-C₆)alkyl, aryl, CON(R37)(R38), N(R39)(R40), O—(C₁-C₆)alkyl, or NHCO(C₁-C₆)alkyl;

X is a bond, C(R8)(R9), O, N(R12), S, or SO₂;

R8, R9, R12 are independently of one another H, or (C₁-C₆)alkyl;

D is N, or C(R41);

E is N, or C(R42);

G is N, or C(R43);

L is N, or C(R44);

where the total number of the nitrogen atoms defined by D, E, G and L is 0, 1 or 2;

R1, R2, R3, R41, R42, R43, R44 are independently of one another H, F, Cl, Br, CF₃, NO₂, O—(C₁-C₆)alkyl, (C₁-C₆)alkyl, (C₃-C₈)cycloalkyl, O—(C₃-C₈)cycloalkyl, (C₂-C₆)alkynyl, (C₀-C₈)alkylenearyl, —O—(C₀-C₃)alkylenearyl, S—aryl, N(R13)(R14), SO₂-CH₃, COO—(C₁-C₆)alkyl, CON(R15)(R16), N(R17)CO(R18), N(R19)SO₂(R20), or CO(R21);

R13, R14 are independently of one another H, (C₁-C₆)alkyl, or R13 and R14 together with the nitrogen atom to which they are bonded form a 5- to 6-membered ring, where, in the case of the 6-membered ring, a CH₂ group may be replaced by O or S;

R15, R16 are independently of one another H, (C₁-C₆)alkyl, or R15 and R16 together with the nitrogen atom to which they are bonded form a 5- to 6-membered ring, where, in the case of the 6-membered ring, a CH₂ group may be replaced by O or S;

R17, R19 are independently of one another H, or (C₁-C₆)alkyl;

R18, R20, R21 are independently of one another (C₁-C₆)alkyl, or aryl;

B is N(R24), or O;

R24 is H, or (C₁-C₆)alkyl;

R5 is H, or (C₁-C₆)alkyl;

W is N, or C(R25);

R25 is H, (C₁-C₆)alkyl, or aryl;

T is C(R26);

R26 is H, (C₁-C₆)alkyl, aryl, or a bond to Y;

U is O, S, N(R27), or —N═C(R31)—;

wherein R27, R31 are independently of one another H, (C₁-C₆)alkyl, or a bond to Y;

Y is (C₁-C₄)alkylene, in which a carbon may be replaced by SO₂, C(R32)(R33), CO or N(R36);

R32, R33, R36 are independently of one another H, (C₁-C₆)alkyl, or aryl;

R6, R7 are independently of one another H, (C₁-C₆)alkyl, (C₃-C₇)cycloalkyl, or R6 and Y or R6 and R7 together with the nitrogen atom to which they are bonded form a 4- to 7-membered ring in which one or more carbons may be replaced by O, N or S and the 4- to 7-membered ring may carry further substituents such as (C₁-C₆)alkyl, aryl, CON(R37)(R38), N(R39)(R40), OH or N HCO(C₁-C₆)alkyl;

R37, R38, R39, R40 are independently of one another H, or (C₁-C₆)alkyl;

and the physiologically acceptable salts thereof.

Particular preference is given to compounds of formula I, in which one or more radicals have the following meaning:

A is (C₃-C₇)alkyl, (C₀-C₂)alkylenearyl; a 5- to 10-membered mono- or bicyclic ring which may contain 0, 1 or 2 heteroatoms selected from the group consisting of N, O and S, and the 5- to 10-membered ring may carry further substituents, such as F, Cl, Br, NO₂, CF₃, (C₁-C₆)alkyl, aryl, O—(C₁-C₆)alkyl or NHCO(C₁-C₆)alkyl;

X is a bond, C(R8)(R9), O, or N(R12);

R8, R9, R12 are independently of one another H, or (C₁-C₆)alkyl;

D is N, or C(R41);

E is N, or C(R42);

G is N, or C(R43);

L is N, or C(R44);

where the total number of the nitrogen atoms defined by D, E, G and L is 0 or 1;

R1, R2, R3, R41, R42, R43, R44 are independently of one another H, F, Cl, CF₃, NO₂, O—(C₁-C₆)alkyl, (C₁-C₆)alkyl, O—(C₃-C₈)cycloalkyl, (C₀-C₂)alkylenearyl, —O—(C₀-C₃)alkylenearyl, N(R13)(R14), COO—(C₁-C₆)alkyl, CON(R15)(R16), N(R17)CO(R18), N(R19)SO₂(R20), or CO(R21);

R13, R14 are independently of one another H, or (C₁-C₆)alkyl,

R15, R16 are independently of one another H, or (C₁-C₆)alkyl,

R17, R19 are independently of one another H, or (C₁-C₆)alkyl;

R18, R20, R21 are independently of one another (C₁-C₆)alkyl, or aryl;

B is N(R24);

R24 is H, or (C₁-C₆)alkyl;

R5 is H, or (C₁-C₆)alkyl;

W is N, or C(R25);

R25 is H, or (C₁-C₆)alkyl;

T is C(R26);

R26 is H, (C₁-C₆)alkyl, or a bond to Y;

U is O, S, or N(R27);

R27 is H, (C₁-C₆)alkyl, or a bond to Y;

Y is (C₁-C₃)alkylene, in which a carbon may be replaced by SO₂, C(R32)(R33) or CO;

R32, R33 are independently of one another H, (C₁-C₆)alkyl, or aryl;

R6, R7 are independently of one another H, (C₁-C₆)alkyl, (C₃-C₇)cycloalkyl, or R6 and Y or R6 and R7 together with the nitrogen to which they are bonded form a 5- to or 6-membered ring in which one or more carbons may be replaced by O or N and the 5- or 6-membered ring may carry further substituents, such as (C₁-C₆)alkyl, aryl, CON(R37)(R38), N(R39)(R40), OH or NHCO(C₁-C₆)alkyl;

R37, R38, R39, R40 are independently of one another H, or (C₁-C₆)alkyl; and the physiologically acceptable salts thereof.

The invention relates to compounds of formula I in the form of their racemates, enantiomer-enriched mixtures and pure enantiomers and to their diastereomers and mixtures thereof.

The substituents R1, R2, R3, R4, R5, R6, R7, R8, R9, R10, R11, R12, R13 R14, R15, R16, R17, R18, R19, R20, R21, R22, R25, R26, R27, R30, R31, R32, R33, R34, R35, R36, R37, R38, R39, R40, R41, R42, R43 and R44 may have straight-chain, branched or optionally halogenated alkyl, alkylene, alkenyl and alkynyl radicals.

The term “aryl” means a phenyl or naphthyl group. The term “ring” means a cyclic structure which may be aromatic, partly saturated or completely saturated. The optional ring formation of R6, Y and the nitrogen to which they are bonded can be illustrated by examples 6 and 16 without limiting the general description mentioned above.

Pharmaceutically acceptable salts are particularly suitable for medical applications, due to their greater solubility in water compared with the starting or base compounds. Said salts must have a pharmaceutically acceptable anion or cation. Suitable pharmaceutically acceptable acid addition salts of the compounds of the invention are salts of inorganic acids, such as hydrochloric acid, hydrobromic acid, phosphoric acid, metaphosphoric acid, nitric acid, sulfonic acid and sulfuric acid and also of organic acids, such as, for example, acetic acid, benzenesulfonic acid, benzoic acid, citric acid, ethanesulfonic acid, fumaric acid, gluconic acid, glycolic acid, isethionic acid, lactic acid, lactobionic acid, maleic acid, malic acid, methanesulfonic acid, succinic acid, p-toluenesulfonic acid, tartaric acid and trifluoroacetic acid. For medicinal purposes, particular preference is given to using the chloride salt. Suitable pharmaceutically acceptable basic salts are ammonium salts, alkali metal salts (such as sodium salts and potassium salts) and alkaline earth metal salts (such as magnesium salts and calcium salts).

Salts having a pharmaceutically unacceptable anion are likewise included within the scope of the present invention as useful intermediates for preparing or purifying pharmaceutically acceptable salts and/or for use in nontherapeutic applications, for example in-vitro applications.

The term “physiologically functional derivative” used herein relates to any physiologically acceptable derivative of an inventive compound of formula I, for example, an ester which on administration to a mammal (e.g., humans) is capable of forming (directly or indirectly) a compound of formula I or an active metabolite thereof.

The physiologically functional derivatives also include prodrugs of the compounds of the invention. Such prodrugs may be metabolized in vivo to a compound of the invention. These prodrugs may or may not be active themselves.

The compounds of the invention may also be present in various polymorphous forms, for example as amorphous and crystalline polymorphous forms. All polymorphous forms of the compounds of the invention are included within the scope of the invention and are another aspect of the invention.

All references to “compound(s) according to formula (I)” refer hereinbelow to a compound/compounds of the formula (I) as described above and also to their salts, solvates and physiologically functional derivatives as described herein.

The amount of a compound according to formula (I) which is required in order to attain the desired biological effect depends on a number of factors, for example the specific compound selected, the intended use, the type of administration and the clinical state of the patient. In general, the daily dose is in the range from 0.3 mg to 100 mg (typically from 3 mg to 50 mg) per day per kilogram of body weight, for example 3-10 mg/kg/day. An intravenous dose can be, for example, in the range from 0.3 mg to 1.0 mg/kg and can be administered in a suitable manner as an infusion of 10 ng to 100 ng per kilogram per minute. Suitable infusion solutions for these purposes may contain, for example, from 0.1 ng to 10 mg, typically from 1 ng to 10 mg per milliliter. Individual doses may contain, for example, from 1 mg to 10 g of the active compound. Thus, ampoules for injections can contain, for example, from 1 mg to 100 mg, and orally administerable individual dose formulations such as, for example, tablets or capsules can contain, for example, from 1.0 to 1000 mg, typically from 10 to 600 mg. In the case of pharmaceutically acceptable salts, the abovementioned masses relate to the mass of the free compound on which the salt is based. The compound used for the prophylaxis or therapy of the abovementioned conditions may be the compounds according to formula (I) themselves, but they are preferably present in the form of a pharmaceutical composition together with an acceptable carrier. The carrier must be naturally acceptable, in the sense that it is compatible with the other ingredients of said composition and is not harmful to the patient's health. The carrier may be a solid or a liquid or both and is preferably formulated with the compound as an individual dose, for example, as a tablet which may contain from 0.05% to 95% by weight of the active compound. Further pharmaceutically active substances may also be present, including further compounds according to formula (I). The pharmaceutical compositions of the invention may be prepared according to any of the known pharmaceutical methods which essentially comprise mixing the ingredients with pharmacologically acceptable carriers and/or excipients.

Pharmaceutical compositions of the invention are those which are suitable for oral, rectal, topical, peroral (e.g., sublingual) and parenteral (e.g., subcutaneous, intramuscular, intradermal or intravenous) administration, although the most suitable manner of administration depends in each individual case on the nature and severity of the condition to be treated and on the nature of the compound according to formula (I) used in each case. Sugar-coated formulations and sugar-coated delayed-release formulations, too, are included within the scope of the invention. Preference is given to acid-resistant and enteric formulations. Suitable enteric coatings include cellulose acetate phthalate, polyvinyl acetate phthalate, hydroxypropylmethylcellulose phthalate and anionic polymers of methacrylic acid and methyl methacrylate.

Suitable pharmaceutical compounds for oral administration may be present in separate units as, for example, capsules, cachets, lozenges or tablets, which in each case contain a particular amount of the compound according to formula (I); as powders or granules; as solution or suspension in an aqueous or nonaqueous liquid; or as an oil-in-water or water-in-oil emulsion. As already mentioned, said compositions can be prepared according to any suitable pharmaceutical method which includes a step in which the active compound and the carrier (which may comprise one or more additional components) are contacted. In general, the compositions are prepared by uniform and homogeneous mixing of the active compound with a liquid and/or finely dispersed solid carrier, after which the product is shaped, if necessary. Thus, a tablet, for example, may be prepared by pressing or shaping a powder or granules of the compound, where appropriate with one or more additional components. Pressed tablets can be prepared by tableting the compound in free-flowing form, for example, a powder or granules, mixed, where appropriate, with a binder, lubricant, inert diluent and/or one or more surface active/dispersing agents in a suitable machine. Shaped tablets can be prepared by shaping the pulverulent compound, moistened with an inert liquid diluent, in a suitable machine.

Pharmaceutical compositions which are suitable for peroral (sublingual) administration include lozenges which contain a compound according to formula (I) with a flavoring, usually sucrose and gum arabic or tragacanth, and pastilles which comprise the compound in an inert base such as gelatin and glycerol or sucrose and gum arabic.

Suitable pharmaceutical compositions for parenteral administration preferably comprise sterile aqueous preparations of a compound according to formula (I) which are preferably isotonic with the blood of the intended recipient. These preparations are preferably administered intravenously, although they may also be administered subcutaneously, intramuscularly or intradermally as an injection. Said preparations may preferably be prepared by mixing the compound with water and rendering the obtained solution sterile and isotonic with the blood. Injectable compositions of the invention generally contain from 0.1 to 5% by weight of the active compound.

Suitable pharmaceutical compositions for rectal administration are preferably present as individual dose suppositories. These may be prepared by mixing a compound according to formula (I) with one or more conventional solid carriers, for example, cocoa butter, and shaping the resulting mixture.

Suitable pharmaceutical compositions for topical application to the skin are preferably present as ointment, cream, lotion, paste, spray, aerosol or oil. Carriers which may be used are petroleum jelly, lanolin, polyethylene glycols, alcohols and combinations of two or more of these substances. In general, the active compound is present at a concentration of from 0.1 to 15%, for example from 0.5 to 2%, by weight of the composition.

Transdermal administration is also possible. Suitable pharmaceutical compositions for transdermal administration may be present as individual patches which are suitable for long-term close contact with the epidermis of the patient. Such patches suitably contain the active compound in an optionally buffered aqueous solution, dissolved and/or dispersed in an adhesive or dispersed in a polymer. A suitable active compound concentration is from approx. 1% to 35%, preferably approx. 3% to 15%. A particular possibility is the release of the active compound by electrotransport or iontophoresis, as described, for example, in Pharmaceutical Research, 2(6):318 (1986).

The compounds of formula I are distinguished by beneficial actions on the metabolism of lipids, and they are particularly suitable for weight reduction and, after weight reduction, for maintaining a reduced weight in mammals and as anorectic agents. The compounds are distinguished by their low toxicity and their few side effects. The compounds may be employed alone or in combination with other weight-reducing or anorectic active compounds. Further anorectic active compounds of this kind are mentioned, for example, in the Rote Liste 2001, Arzneimittelverzeichnis fur Deutschland, Rote Liste Service GmbH, Frankfurt, under weight-reducing agents/appetite suppressants, and may also include those active compounds which increase the energy turnover of the organism and thus lead to weight reduction or else those which influence the general metabolism of said organism such that increased calorie intake does not cause an enlargement of the fat depots and a normal calorie intake causes a reduction in the fat depots of said organism. The compounds are suitable for the prophylaxis and, in particular, for the treatment of problems of excess weight or obesity. The compounds are furthermore suitable for the prophylaxis and, in particular, for the treatment of type II diabetes, of arteriosclerosis and for the normalization of lipid metabolism and for the treatment of high blood pressure. The compounds act as MCH antagonists and are also suitable for the treatment of paresthesia and other psychiatric indications such as, for example, depressions, anxieties, anxiety neuroses, schizophrenia and also for the treatment of disorders associated with the circadian rhythm and for the treatment of drug abuse.

In a further aspect of the invention, the compounds of formula I may be administered in combination with one or more further pharmacologically active substances which may be selected, for example, from the group consisting of antidiabetics, antiadipose agents, blood-pressure-lowering active compounds, lipid reducers and active compounds for the treatment and/or prevention of complications caused by diabetes or associated with diabetes.

Suitable antidiabetics include insulins, amylin, GLP-1 and GLP-2 derivatives such as, for example, those disclosed by Novo Nordisk A/S in WO 98/08871 and also oral hypoglycemic active compounds.

Said oral hypoglycemic active compounds preferably include sulfonyl ureas, biguanidines, meglitinides, oxadiazolidinediones, thiazolidinediones, glucosidase inhibitors, glucagon receptor antagonists, GLP-1 agonists, potassium channel openers such as, for example, those disclosed by Novo Nordisk A/S in WO 97/26265 and WO 99/03861, insulin sensitizers, activators of insulin receptor kinase, inhibitors of liver enzymes involved in the stimulation of gluconeogenesis and/or glycogenolysis, for example glycogen phosphorase inhibitors, modulators of glucose uptake and glucose elimination, lipid metabolism-modifying compounds such as antihyperlipidemic active compounds and antilipidemic active compounds, for example HMGCoA-reductase inhibitors, inhibitors of cholesterol transport/cholesterol uptake, inhibitors of the reabsorption of bile acid or inhibitors of microsomal triglyceride transfer protein (MTP), compounds which reduce food intake, PPAR and RXR agonists and active compounds which act on the ATP-dependent potassium channel of beta cells.

In one embodiment of the present invention, the present compounds are administered in combination with insulin.

In another embodiment, the compounds of the invention are administered in combination with a sulfonylurea such as, for example, tolbutamide, glibenclamide, glimepiride, glipizide, gliquidone, glisoxepide, glibornuride or gliclazide.

In another embodiment, the compounds of the present invention are administered in combination with a biguanidine such as, for example, metformin.

In another embodiment, the compounds of the present invention are administered in combination with a meglitinide such as, for example, repaglinide.

In yet another embodiment, the compounds of the present invention are administered in combination with a thiazolidinedione such as, for example, troglitazone, ciglitazone, pioglitazone, rosiglitazone or the compounds disclosed by Dr. Reddy's Research Foundation in WO 97/41097, in particular 5-[[4-[(3,4-dihydro-3-methyl-4-oxo-2-quinazolinylmethoxy]phenyl]methyl]-2,4-thiazolidinedione.

In another embodiment, the compounds of the present invention are administered in combination with an α-glucosidase inhibitor such as, for example, miglitol or acarbose.

In another embodiment, the compounds of the present invention are administered in combination with an active compound which acts on the ATP-dependent potassium channel of the beta cells, such as, for example, tolbutamide, glibenclamide, glimepiride, glipizide, gliclazide or repaglinide.

In yet another embodiment, the compounds of the present invention are administered in combination with an antihyperlipidemic active compound or an antilipidemic active compound such as, for example, cholestyramine, colestipol, clofibrate, fenofibrate, gemfibrozil, lovastatin, pravastatin, simvastatin, atorvastatin, cerivastatin, fluvastatin, probucol, ezetimibe or dextrothyroxine.

In another embodiment, the compounds of the present invention are administered in combination with more than one of the aforementioned compounds, for example in combination with a sulfonylurea and metformin, a sulfonylurea and acarbose, repaglinide and mefformin, insulin and a sulfonylurea, insulin and mefformin, insulin and troglitazone, insulin and lovastatin, etc.

Furthermore, the compounds of the invention may be administered in combination with one or more antiadipose agents or appetite-controlling active compounds.

Such active compounds may be selected from the group consisting of CART agonists, NPY antagonists, MC4 agonists, orexin antagonists, H3 agonists, TNF agonists, CRF agonists, CRF BP antagonists, urocortin agonists, β3 agonists, MSH (melanocyte-stimulating hormone) agonists, CCK agonists, serotonin re-uptake inhibitors, mixed serotonin and noradrenalin reuptake inhibitors, 5HT modulators, MAO inhibitors, bombesin agonists, galanin antagonists, growth hormone, growth-hormone-releasing compounds, TRH agonists, uncoupling protein 2 or 3 modulators, leptin agonists, dopamine agonists (bromocriptine, doprexin), lipase/amylase inhibitors, cannabinoid receptor 1 antagonists, modulators of acylation-stimulating protein (ASP), PPAR modulators, RXR modulators, hCNTF mimetics or TR-β agonists.

In one embodiment of the invention, the antiadipose agent is leptin or modified leptin.

In another embodiment, the antiadipose agent is dexamphetamine or amphetamine.

In another embodiment, the antiadipose agent is fenfluramine or dexfenfluramine.

In yet another embodiment, the antiadipose agent is sibutramine or the mono- and bis-demethylated active metabolite of sibutramine.

In another embodiment, the antiadipose agent is orlistate.

In another embodiment, the antiadipose agent is mazindol, diethylpropione or phentermine.

Furthermore, the compounds of the present invention may be administered in combination with one or more antihypertensive active compounds. Examples of antihypertensive active compounds are beta blockers such as alprenolol, atenol, timolol, pindolol, propanolol and metoprolol, ACE (angiotensin-converting enzyme) inhibitors such as, for example, beiiazepril, captopril, enalapril, fosinopril, lisinopril, quinapril and rampril, calcium channel blockers such as nifedipine, felodipine, nicardipine, isradipine, nimodipine, diltiazem and verapamil, and also alpha blockers such as doxazosin, urapidil, prazosin and terazosin. Furthermore, reference may be made to Remington: The Science and Practice of Pharmacy, 19th edition, Gennaro, editor, Mack Publishing Co., Easton, PA, 1995.

It is self-evident that every suitable combination of the compounds of the invention with one or more of the aforementioned compounds and optionally one or more other pharmacologically active substances is to be regarded as covered by the scope of protection of the present invention.

### EXAMPLES

The Activity of the Compounds was Assayed as Follows:

Biological Test Model:

The anorectic action was tested on female NMRI mice. After removal of feed for 17 hours, the preparation to be tested was administered by gavage. The animals were housed singly and, with free access to drinking water, they were offered evaporated milk 30 minutes after administration of the preparation. The consumption of evaporated milk was determined and the general behavior of the animals were monitored every half an hour for 7 hours. The measured milk consumption was compared to that of vehicle-treated control animals.

TABLE 1
Anorectic action, measured as a reduction in
the cumulative milk consumption by treated
animals compared with control animals

| | | Number | Number | Reduction in |
|---|---|---|---|---|
| | | of animals/ | of animals/ | cumulative |
| | | cumulative milk | cumulative milk | milk |
| | Oral | consumption by | consumption by | consumption |
| | dose | treated animals | control animals | as % of |
| Example | [mg/kg] | N/[mL] | N/[mL] | the control |
| Example 1 | 30 | 5/2.28 | 5/3.26 | 30 |
| Example 4 | 10 | 5/2.74 | 5/4.44 | 38 |

The table indicates that the compounds of formula I exhibit very good anorectic action.

In two simultaneously published articles in Nature (Nature, 400:261-264, 1999; Nature, 400:265-269,1999, see enclosure), two groups separately described a highly specific receptor for melanin-concentrating hormone (MCH). MCH takes over important functions in the control of food intake. Compounds acting on the MCH receptor therefore have anorectic action and are suitable for the treatment of obesity. The test for anorectic action of the inventive compounds of formula I was therefore carried out as follows.

Functional Measurements for Determination of IC50

Cloning of the cDNA for human MCH receptor, preparation of a recombinant HEK293 cell line expressing human MCH receptor and functional measurements with said recombinant cell line were carried out according to the description by Audinot et al. (J. Bioi. Chem., 276, 13554-13562, 2001). In contrast to the reference, however, plasmid pEAK8 from EDGE Biosystems (USA) was used for constructing the expression vector. A transformed HEK cell line named “PEAK Stable Cell”(likewise from EDGE Biosystems) served as host for transfection. The functional measurements of cellular calcium flow, after addition of agonists (MCH), in the presence of the ligand of the invention was carried out with the aid of the FLIPR instrument from Molecular Devices (USA), using the manufacturer's protocols.

TABLE 2
Test for anorectic action of the
inventive compounds of formula I;
results from the cellular assay

| Example | IC50/μM |
|---|---|
| 1 | 0.15 |
| 2 | 0.15 |
| 3 | 0.29 |
| 4 | 0.13 |
| 5 | 0.50 |
| 6 | 2.34 |
| 7 | 0.45 |
| 8 | 1.90 |
| 9 | 0.10 |
| 10 | 0.11 |
| 11 | 0.14 |
| 13 | 2.50 |
| 14 | 0.30 |
| 15 | 0.18 |
| 16 | 0.33 |
| 17 | 2.14 |
| 18 | 1.04 |
| 19 | 0.70 |
| 22 | 4.42 |
| 24 | 0.86 |
| 26 | 0.92 |
| 29 | 2.91 |
| 33 | 1.24 |
| 63 | 0.57 |
| 65 | 0.50 |
| 71 | 2.65 |
| 72 | 0.32 |
| 73 | 0.14 |
| 76 | 4.25 |
| 77 | 0.70 |
| 78 | 2.75 |
| 79 | 2.13 |
| 80 | 3.36 |
| 81 | 2.69 |
| 84 | 0.40 |
| 86 | 2.78 |
| 105 | 1.0 |
| 106 | 0.20 |
| 107 | 1.0 |
| 108 | 0.43 |
| 109 | 1.29 |

The examples and preparation methods listed below serve to illustrate the invention but without limiting it.

### Example 1

1-[1-(2-Dimethylaminoethyl)-1 H-indol-5-yl]-3-(4-phenoxyphenyl)urea

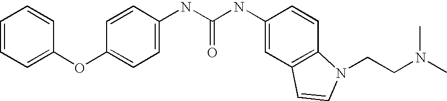

Carbonyldiimidazole (5.12 g) was added to a solution cooled to 0° C. of 1-dimethylaminoethyl-5-aminoindole (6.30 g) in dimethylformamide (50 mL). After 10 minutes 4-aminodiphenyl ether (5.84 g) was added and the reaction mixture was heated to 80° C. for 2 hours. After cooling, the reaction was diluted with ethyl acetate and washed with water. The organic phase was dried over magnesium sulfate, filtered and concentrated. The residue was purified by chromatography on silica gel (eluent: dichloromethane/methanol 9:1). Thus the product having a molecular weight of 414.15 (C₂₅H₂₆N₄O₂); MS (ESI): 415 (M+H⁺) was obtained.

### Example 2

1-(4-Butoxyphenyl)-3-[1-(2-dimethylaminoethyl)-1H-indol-5-yl]urea

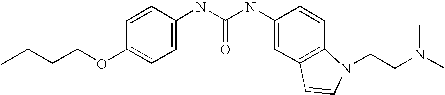

The compound was prepared from 4-butoxyaniline and 1-dimethylaminoethyl-5-aminoindole, as described in Example 1. Thus, the product having a molecular weight of 394.52 (C₂₃H₃₀N₄O₂); MS (ESI): 395 (M+H⁺) was obtained.

### Example 3

1-(1-Methyl-2-pyrrolidin-1-ylmethyl-1H-indol-5-yl)-3-(4-phenoxyphenyl)urea

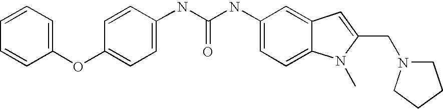

The compound was prepared from 4-aminodiphenyl ether and 1-methyl-2-pyrrolidin-1-ylmethyl-1H-indol-5-ylamine, as described in Example 1. Thus, the product having a molecular weight of 440.55 (C₂₇H₂₈N₄O₂); MS (ESI): 441 (M+H₊) was obtained.

1-Methyl-2-pyrrolidin-1-ylmethyl-1H-indol-5-ylamine

Formic acid (0.11 mL) was added to a suspension of 1-methyl-5-nitro-2-pyrrolidin-1-ylmethyl-1H-indole (150 mg), ethanol (2 mL) and palladium(II) hydroxide on carbon (20%, 30 mg) and the suspension was heated to 60° C. for 5 minutes. After gas production had ceased, the suspension was stirred for another 20 minutes and the catalyst was filtered off. The filtrate was concentrated and distributed between saturated sodium carbonate solution and methyl tert-butyl ether. The organic phase was removed, dried over magnesium sulfate and concentrated. Thus, the product having a molecular weight of 229.33 (C₁₄H₁₉N₃); MS (ESI): 230 (M+H⁺) was obtained.

1-Methyl-5-nitro-2-pyrrolidin-1-ylmethyl-1H-indole

Mesyl chloride (92 mg) was added dropwise to a solution cooled to 0° C. of (1-methyl-5-nitro-1H-indol-2-yl)methanol (121 mg) in dichloromethane (10 mL) and triethylamine (0.17 mL). After 15 minutes, pyrrolidine (142 mg) was added and the solution was then stirred at room temperature for 1 hour. The reaction solution was washed with saturated sodium carbonate solution, dried over magnesium sulfate and concentrated. The residue was purified via chromatography on silica gel (eluent: ethyl acetate/triethylamine 99:1). Thus, the product having a molecular weight of 259.31 (C₁₄H₁₇N₃O₂); MS (ESI): 260 (M+H⁺) was obtained.

(1-Methyl-5-nitro-1H-indol-2-yl)methanol

Sulfuric acid (96% strength, 0.64 mL) was added dropwise to a suspension cooled to 0° C. of lithium aluminum hydride in tetrahydrofuran (50 mL) within 20 minutes. After 20 minutes, a solution of ethyl 1-methyl-5-nitro-1H-indole 2-carboxylate (1.85 g) in tetrahydrofuran (40 mL) was added dropwise. After 30 minutes, water (2 mL) was added. After 30 minutes, the resulting precipitate was filtered off and the filtrate was concentrated. The crude product was purified via chromatography on silica gel (eluent: n-heptane/ethyl acetate 3:2). Thus, the product having a molecular weight of 206.20 (C₁₀H₁₀N₂O₃); MS (ESI): 207 (M+H⁺) was obtained.

Ethyl 1-methyl-5-nitro-1H-indole 2-carboxylate

A suspension of ethyl 5-nitro-1H-indole 2-carboxylate (2.34 g), potassium carbonate (3.45 g), methyl iodide (2.13 g) and acetonitrile (30 mL) was kept at 60° C. for 6 hours. After cooling to room temperature, water was added and the precipitated product was isolated by filtration. Thus, the product having a molecular weight of 248.24 (C₁₂H₁₂N₂O₄); MS (ESI): 249 (M+H⁺) was obtained.

### Example 4

1-[1-(2-Dimethylaminoethyl)-1H-benzoimidazol-5-yl]-3-(4-phenoxyphenyl)urea

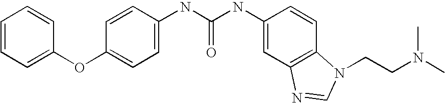

Zinc dust (250 mg) was added to a solution of 1-[4-(2-dimethylaminoethylamino)-3-nitrophenyl]-3-(4-phenoxyphenyl)urea (50 mg) in dichloromethane (10 mL) and glacial acetic acid (1 mL). After 10 minutes, the inorganic material was filtered off via kieselguhr. The filtrate was washed with a sodium carbonate solution (10% strength), dried over magnesium sulfate and concentrated. The residue was taken up in dichloromethane (5 mL) and ethanol (5 mL) and admixed with dimethylformamide dimethyl acetal (0.3 mL) and formic acid (0.3 mL). Dichloromethane was evaporated by heating the mixture by means of a hot-air gun. The remaining mixture was concentrated and distributed between dichloromethane and a sodium carbonate solution (10% strength). The organic phase was removed, dried and concentrated. The residue was purified by preparative HPLC. Thus, the product having a molecular weight of 415.50 (C₂₄H₂₅N₅O₂); MS (ESI): 416 (M+H⁺) was obtained. Melting point of the hydrochloride: 213-215° C.

1-[4-(2-Dimethylaminoethylamino)-3-nitrophenyl]-3-(4-phenoxyphenyl)urea

A solution of 2-dimethylaminoethylamine in dimethylformamide (1 M, 2 mL) and 1-(4-fluoro-3-nitrophenyl)-3-(4-phenoxyphenyl)urea (200 mg) was stirred for 48 hours. The mixture was distributed between dichloromethane and a sodium carbonate solution (10% strength). The organic phase was dried and concentrated. The residue was recrystallized from toluene. Melting point: 178-180° C.

1-(4-Fluoro-3-nitrophenyl)-3-(4-phenoxyphenyl)urea

4-Fluoro-3-nitrophenyl isocyanate (2.2 mmol) was added to a solution of 4-phenoxyaniline (2 mmol) in dimethylformamide (20 mL). After 2 days, the reaction mixture was distributed between dichloromethane and a saturated sodium carbonate solution. The organic phase was dried and concentrated. The residue was purified via chromatography on silica gel (eluent: ethyl acetate/dichloro-methane 95:5) and subsequent recrystallization from ethyl acetate/hexane. Melting point: 174-176° C.

### Example 5

1-[1-(2-Dimethylaminoethyl)-2-methyl-1H-benzoimidazol-5-yl]-3-(4-isopropoxyphenyl)urea

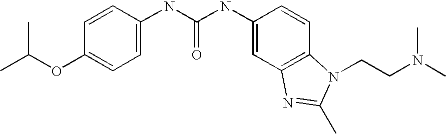

1-[4-(2-Dimethylaminoethylamino)-3-nitrophenyl]-3-(4-isopropoxyphenyl)urea (75 mg) was reduced using zinc dust, as described in Example 4. The reaction product was dissolved in methanol and admixed with triethyl orthoacetate (0.5 mL) and glacial acetic acid (0.2 mL). The mixture was heated under reflux for 5 minutes. Volatile components were removed. The residue was distributed between dichloromethane and a sodium carbonate solution. The organic phase was dried and concentrated. The residue was purified by preparative HPLC. Thus, the product having a molecular weight of 395.51 (C₂₂H₂₉N₅O₂); MS (ESI): 396 (M+H⁺) was obtained.

1[-4-(2-Dimethylaminoethylamino)-3-nitrophenyl]-3-(4-isopropoxyphenyl)urea

The compound was obtained from 1-(4-fluoro-3-nitrophenyl)-3-(4-isopropoxyphenyl)urea and 2-dimethylaminoethylamine as in Example 4. The compound was reacted further without purification.

1-(4-Fluoro-3-nitrophenyl)-3-(4-isopropoxyphenyl)urea

The compound was obtained from 4-fluoro-3-nitrophenyl isocyanate and 4-isopropoxyaniline as in Example 4. Melting point: 170-172° C.

### Example 6

1-[1-(1-Ethylpyrrolidin-2-ylmethyl)-2-methyl-1H-benzoimidazol-5-yl]-3-(4-isopropoxyphenyl)urea

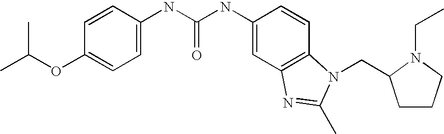

The compound was prepared from 1-{4-[(1-ethylpyrrolidin-2-ylmethyl)amino]-3-nitrophenyl}-3-(4-isopropoxyphenyl)urea, as described in Example 5. Thus, the product having a molecular weight of 435.57 (C₂₅H₃₃N₅O₂); MS (ESI): 436 (M+H⁺) was obtained. Melting point: (ethyl acetate/hexane): 185-187° C.

1-{4-[(1-Ethylpyrrolidin-2-ylmethyl)amino]-3-nitrophenyl}-3-(4-isopropoxy-phenyl)urea

The compound was prepared from 1-(4-fluoro-3-nitrophenyl)-3-(4-isopropoxyphenyl)urea and 1-ethylpyrrolidin-2-ylmethylamine, as described in Example 4, and reacted further without any further purification.

### Example 7

1-(4-Isopropoxyphenyl)-3-[2-methyl-1-(2-piperidin-1-ylethyl)-1H-benzoimidazol-5-yl]urea

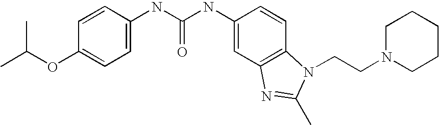

The compound was prepared from 1-(4-isopropoxyphenyl)-3-[3-nitro-4-(2-piperidin-1-yl-ethylamino)phenyl]urea, as described in Example 5. Thus the product having a molecular weight of 435.57 (C₂₅H₃₃N₅O₂); MS (ESI): 436 (M+H⁺) was obtained.

1-(4-Isopropoxyphenyl)-3-[3-nitro-4-(2-piperidin-1-ylethylamino)phenyl]urea

The compound was prepared from 1-(4-fluoro-3-nitrophenyl)-3-(4-isopropoxyphenyl)urea and 1-(2-aminoethyl)piperidine (60° C., 4 h), as described in Example 4. Melting point (ethyl acetate): 157-159° C.

### Example 8

1-(4-Isopropoxyphenyl)-3-[2-methyl-1-(2-morpholin-4-yl-ethyl)-1H-benzoimidazol-5-yl]urea

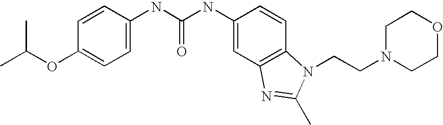

The compound was prepared from 1-(4-isopropoxyphenyl)-3-[4-(2-morpholin-4-ylethylamino)-3-nitrophenyl]urea, as described in Example 5. Thus, the product having a molecular weight of 437.55 (C₂₄H₃₁N₅O₃); MS (ESI): 438 (M+H⁺) was obtained.

1-Isopropoxyphenyl)-3-[4-(2-morpholin-4-yl-ethylamino)-3-nitrophenyl]urea

The compound was prepared from 1-(4-fluoro-3-nitrophenyl)-3-(4-isopropoxyphenyl)urea and 1-(2-aminoethyl)morpholine (60° C., 4 h), as described in Example 4. Melting point (ethyl acetate): 191-193° C.

### Example 9

1 (4-Isopropoxyphenyl)-3-[2-methyl-1-(2-pyrrolidin-1-ylethyl)-1H-benzoimidazol-5-yl]urea

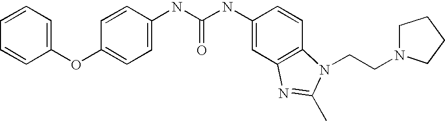

The compound was prepared from 1-[3-nitro-4-(2-pyrrolidin-1-ylethylamino)-phenyl]-3-(4-phenoxyphenyl)urea, as described in Example 5. Thus, the product having a molecular weight of 455.56 (C₂₇H₂₉N₅O₂); MS (ESI): 456 (M+H⁺) was obtained.

1-[3-Nitro-4-(2-pyrrolidin-1-ylethylamino)phenyl]-3-(4-phenoxyphenyl)urea

The compound was prepared from 1-(4-fluoro-3-nitrophenyl)-3-(4-phenoxy-phenyl)urea and 1-(2-aminoethyl)pyrrolidine (60° C., 5 h), as described in Example 4. Melting point (ethyl acetate/hexane): 179-181° C.

### Example 10

1-[2-Methyl-1-(2-dimethylaminoethyl)-1H-benzoimidazol-5-yl]-3-(4-phenoxyphenyl)urea

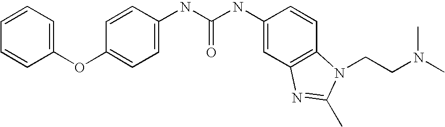

The compound was prepared from 1-[4-(2-dimethylaminoethylamino)-3-nitrophenyl]-3-(4-phenoxyphenyl)urea, as described in Example 5. Thus, the product having a molecular weight of 429.53 (C₂₅H₂₇N₅O₂); MS (ESI): 430 (M+H⁺) was obtained.

### Example 11

1-(4-Phenoxyphenyl)-3-[1-(2-pyrrolidin-1-ylethyl)-1H-benzoimidazol-5-yl]urea

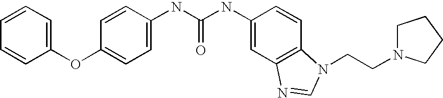

The compound was prepared from 1-[3-nitro-4-(2-pyrrolidin-1-ylethylamino)-phenyl]-3-(4-phenoxyphenyl)urea, as described in Example 4. Thus, the product having a molecular weight of 441.54 (C₂₆H₂₇N₅O₂); MS (ESI): 442 (M+H⁺) was obtained.

### Example 12

1-[2-Benzyl-1-(2-dimethylaminoethyl)-1H-benzoimidazol-5-yl]-3-(4-phenoxyphenyl)urea

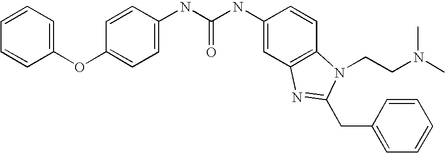

1-[4-(2-Dimethylaminoethylamino)-3-nitrophenyl]-3-(4-phenoxyphenyl)urea (75 mg) was reduced as described in Example 4. The crude product was treated with phenylacetic acid (0.33 mmol), activated with HATU (0.33 mmol), and diisopropylamine (0.7 mmol) in dimethylformamide (1.5 mL) for 3 hours. The reaction mixture was distributed between dichloromethane and a sodium carbonate solution (10% strength). The organic phase was dried and concentrated. The residue was heated under reflux in trifluoroacetic acid (1 mL), water (1 mL) and acetonitrile (0.5 mL) for 5 minutes. Volatile components were evaporated and the residue was purified by preparative HPLC. Thus, the product having a molecular weight of 505.63 (C₃₁H₃₁N₅O₂); MS (ESI): 506 (M+H⁺) was obtained.

### Example 13

1-[1-(2-Dimethylaminoethyl)-2-phenyl-1H-benzoimidazol-5-yl]-3-(4-phenoxyphenyl)urea

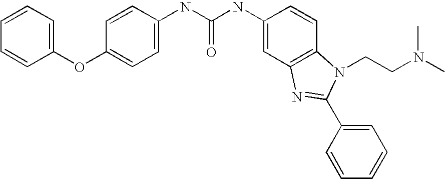

1-[4-(2-Dimethylaminoethylamino)-3-nitrophenyl]-3-(4-phenoxyphenyl)urea (50 mg) was reduced as described in Example 4. After filtration via kieselguhr, benzaldehyde (0.2 mL) was added to the filtrate. The reaction mixture was washed with a sodium carbonate solution (10% strength), dried and admixed with manganese dioxide (0.5 g). After 15 minutes, the inorganic material was filtered off and the filtrate was concentrated. The crude product was purified by preparative HPLC. Thus, the product having a molecular weight of 491.60 (C₃₀H₂₉N₅O₂); MS (ESI): 492 (M+H⁺) was obtained.

### Example 14

1-[2-Ethyl-1-(2-pyrrolidin-1-ylethyl)-1H-benzoimidazol-5-yl]-3-(4-phenoxyphenyl)urea

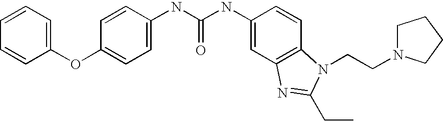

1-[4-(2-Pyrrolidinoethylamino)-3-nitrophenyl]-3-(4-phenoxyphenyl)urea was reduced as described in Example 4. The crude product was reacted with triethyl orthopropionate according to Example 5. The crude product was purified by preparative HPLC. Thus, the product having a molecular weight of 469.59 (C₂₈H₃₁N₅O₂); MS (ESI): 470 (M+H⁺) was obtained.

### Example 15

1-[2-Methyl-1-(2-piperidin-1-ylethyl)-1H-benzoimidazol-5-yl]-3-(4-phenoxyphenyl)urea

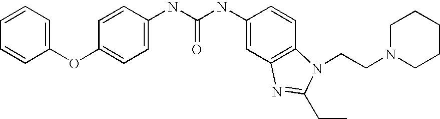

1-[2-Methyl-1-(2-piperidin-1-ylethyl)-1H-benzoimidazol-5-yl]-3-(4-phenoxyphenyl)urea was reduced as described in Example 4. The crude product was reacted with triethyl orthoacetate, as described in Example 5. The crude product was purified by preparative HPLC. Thus, the product having a molecular weight of 469.59 (C₂₈H₃₁N₅O₂); MS (ESI): 470 (M+H⁺) was obtained.

1-[2-Methyl-1-(2-piperidin-1-ylethyl)-1H-benzoimidazol-5-yl]-3-(4-Phenoxyphenyl)-urea

The compound was prepared from 1-(4-fluoro-3-nitrophenyl)-3-(4-phenoxy-phenyl)urea and 1-(2-aminoethyl)piperidine (60° C., 4 h), as described in Example 4. Melting point (ethyl acetate/hexane): 163-165° C.

### Example 16

1-[1-(1-Ethylpyrrolidin-2-ylmethyl)-2-methyl-1H-benzoimidazol-5-yl]-3-(4-phenoxy-phenyl)urea

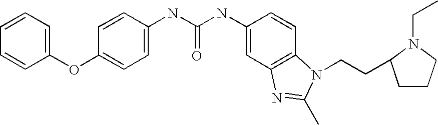

1-{4-[(1-Ethylpyrrolidin-2-ylmethyl)amino]-3-nitrophenyl}-3-(4-phenoxyphenyl)urea was reduced as described in Example 4. The crude product was reacted with triethylorthoacetate, as described in Example 5. The crude product was purified by preparative HPLC. Thus, the product having a molecular weight of 469.59 (C₂₈H₃₁N₅O₂); MS (ESI): 470 (M+H⁺) was obtained.

1-{4-[(1-Ethylpyrrolidin-2-ylmethyl)amino]-3-nitrophenyl}-3-(4-phenoxyphenyl)urea

The compound was prepared from 1-(4-fluoro-3-nitrophenyl)-3-(4-phenoxyphenyl)urea and C-(1-ethylpyrrolidin-2-yl)methylamine (60° C., 4 h), as described in Example 4. Melting point (ethyl acetate/hexane): 129-132° C.

### Example 17

1-(2-Dimethylaminomethyl-1H-benzoimidazol-5-yl)-3-(4-phenoxyphenyl)urea

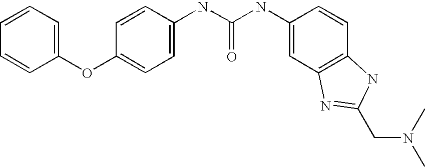

1-[4-(2,4-Dimethoxybenzylamino)-3-nitrophenyl]-3-(4-phenoxyphenyl)urea (75 mg) was reduced as described in Example 4. The reduced product was reacted with dimethylaminoacetic acid (1 mmol), HATU (1 mmol) and diisopropylamine (2 mmol) in dimethylformamide (3 mL). After 3 hours, the mixture was distributed between ethyl acetate and a sodium carbonate solution. The organic phase was dried and concentrated. The crude product was purified by preparative HPLC. Thus the intermediate (N-{2-amino-5-[3-(4-phenoxyphenyl)ureido]phenyl}-2-dimethylaminoacetamide) having a molecular weight of 419.49 (C₂₃H₂₅N₅O₃); MS (ESI): 420 (M+H⁺) was obtained.

This material was heated under reflux with pivalic acid and volatile components were then removed under a high vacuum. The crude product was purified by preparative HPLC. Thus, the product having a molecular weight of 401.47 (C₂₃H₂₃N₅O₂); MS (ESI): 402 (M+H⁺) was obtained.

1-[4-(2,4-Dimethoxybenzylamino)-3-nitrophenyl]-3-(4-phenoxyphenyl)urea

The compound was prepared from 1-(4-fluoro-3-nitrophenyl)-3-(4-phenoxyphenyl)urea and 2,4-dimethoxybenzylamine (60° C., 12 h), as described in Example 4. Melting point (ethyl acetate): 214-216° C.

### Example 18

1-[1-(2-Dimethylaminoethyl)-2,3-dimethyl-1H-indol-5-yl]-3-(4-phenoxyphenyl)urea

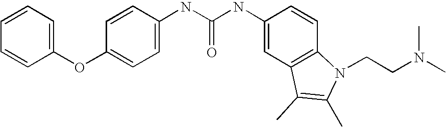

The compound was prepared from 1-(2-dimethylaminoethyl)-2,3-dimethyl-1H-indol-5-ylamine and 4-phenoxyaniline, as described in Example 1. The crude product was purified by preparative HPLC. Thus, the product having a molecular weight of 442.57 (C₂₇H₃₀N₄O₃); MS (ESI): 443 (M+H⁺) was obtained.

1-(2-Dimethylaminoethyl)-2.3-dimethyl-1H-indol-5-ylamine

The compound was obtained by hydrogenation of [2-(2,3-dimethyl-5-nitroindol-1-yl)ethyl]dimethylamine, as described in Example 3. Thus, the product having a molecular weight of 231.34 (C₁₄H₂₁N₃); MS (ESI): 232 (M+H⁺) was obtained.

[2-(2,3-Dimethyl-5-nitroindol-1-yl]dimethylamine

Sodium hydride (50% strength in oil; 0.8 g) was added to 2,3-dimethyl-5-nitro-1H-indole (1 g) in tetrahydrofuran (10 mL) at 0° C. After 30 minutes at room temperature, dimethylaminoethyl chloride (hydrochloride; 1.1 g) was added and the mixture was then heated at 65° C. for two hours. The cooled reaction solution was extracted with dichloromethane. The organic phase was dried and concentrated. The crude product was purified via chromatography on silica gel (eluent: dichloromethane/methanol 9:1). Thus, the product having a molecular weight of 261.33 (C₁₄H₁₉N₃O₂); MS (ESI): 262 (M+H⁺) was obtained.

### Example 19

1-[1-(2-Dimethylaminoethyl)-2-methyl-1H-indol-5-yl]-3-(4-phenoxyphenyl)urea

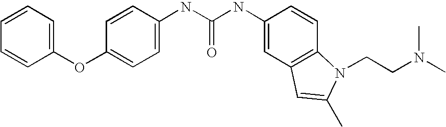

The compound was prepared from 1-(2-dimethylaminoethyl)-2-methyl-1H-indol-5-ylamine and 4-phenoxyaniline, as described in Example 1. The crude product was purified by preparative HPLC. Thus, the product having a molecular weight of 428.54 (C₂₆H₂₈N₄O₃); MS (ESI): 428 (M+H⁺) was obtained.

1-(2-Dimethylaminoethyl)-2-methyl-1H-indol-5-ylamine

The compound was obtained by hydrogenation of [2-(2-methyl-5-nitroindol-1-yl)ethyl]dimethylamine, as described in Example 3. Thus, the product having a molecular weight of 217.32 (C₁₃H₁₉N₃); MS (ESI): 218 (M+H⁺) was obtained.

[2-(2-Methyl-5-nitroindol-1-yl)ethyl]dimethylamine

The compound was prepared from 2-methyl-5-nitro-1H-indole and dimethyl-aminoethyl chloride (hydrochloride) as in Example 18. Thus, the product having a molecular weight of 247.30 (C₁₃H₁₇N₃O₂); MS (ESI): 248 (M+H⁺) was obtained. Table 3: Examples of formula I

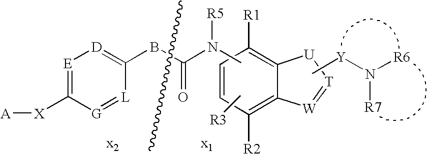

where the moiety x₁ is

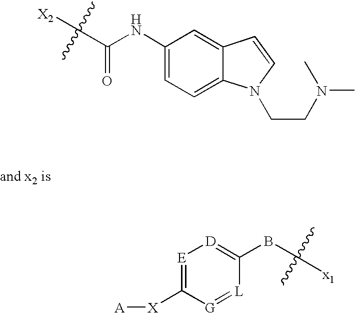

and x₂ is listed in the column denoted “aniline” of the table below.

| Ex- | | | | Mol- | |
|---|---|---|---|---|---|
| am- | | | Molecular | ecular | |
| ple | Name | Aniline | formula | weight | [M + H]+ |
| 20 | 1-[4-(Cyclo- hexylmethyl- amino)phenyl]-3-[1-(2-di- methylaminoethyl)-1H-in- dol-5-yl]urea | 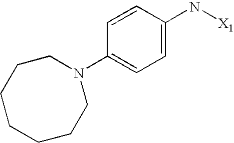 | C26H35N5O | 433.60 | 434 |
| 21 | 1-[4-(Cyclo- hexylmethyl- amino)phenyl]-3-[1-(2-di- methylaminoethyl)-1H-in- dol-5-yl]urea | 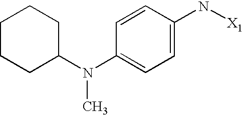 | C26H35N5O | 433.60 | 434 |
| 22 | 1-[1-(2-Di- methylaminoethyl)-1H-in- dol-5-yl]-3-(4-pyr- rolidin-1-ylphe- nyl)urea | 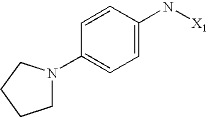 | C23H29N5O | 391.52 | 392 |
| 23 | 1-[1-(2-Di- methylaminoethyl)-1H-in- dol-5-yl]-3-[4-(2,5-di- methylpyrrolidin-1-yl)phe- nyl]urea | 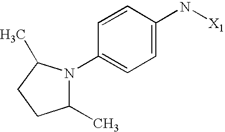 | C25H33N5O | 419.57 | 420 |
| 24 | 1-[4-(3,6-Dihydro-2H-py- ridin-1-yl)phenyl]-3-[1-(2-di- methylaminoethyl)-1H-in- dol-5-yl]urea | 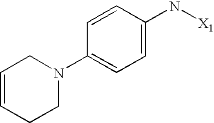 | C24H29N5O | 403.53 | 404 |
| 25 | 1-[1-(2-Di- methylaminoethyl)-1H-in- dol-5-yl]-3-[4-(2,6-di- methylmorpholin-4-yl)phe- nyl]urea | 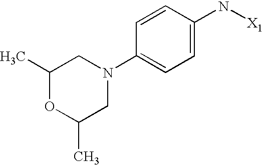 | C25H33N5O2 | 435.57 | 436 |
| 26 | 1-[1-(2-Di- methylaminoethyl)-1H-in- dol-5-yl]-3-(4-thio- morpholin-4-yl-phe- nyl)urea | 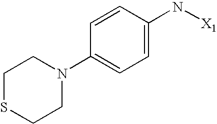 | C23H29N5OS | 423.58 | 424 |
| 27 | 1-[1-(2-Di- methylaminoethyl)-1H-in- dol-5-yl]-3-[4-(2-methyl- piperidin-1-yl)phe- nyl]urea | 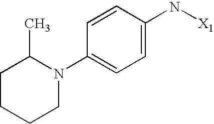 | C25H33N5O | 419.57 | 420 |
| 28 | 1-[1-(2-Di- methylaminoethyl)-1H-in- dol-5-yl]-3-[4-(2-ethyl- piperidin-1-yl)phe- nyl]urea | 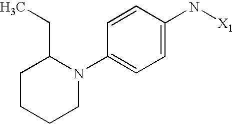 | C26H35N5O | 433.60 | 434 |
| 29 | 1-[1-(2-Di- methylaminoethyl)-1H-in- dol-5-yl]-3-[4-(3-methyl- piperidin-1-yl)phe- nyl]urea | 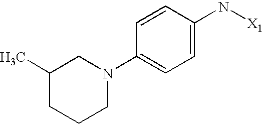 | C25H33N5O | 419.57 | 420 |
| 30 | 1-[1-(2-Di- methylaminoethyl)-1H-in- dol-5-yl]-3-[4-(3,3-di- methylpiperidin-1-yl)phe- nyl]urea | 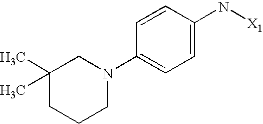 | C26H35N5O | 433.60 | 434 |
| 31 | 1-[1-(2-Di- methylaminoethyl)-1H-in- dol-5-yl]-3-[4-(3,5-di- methylpiperidin-1-yl)phe- nyl]urea | 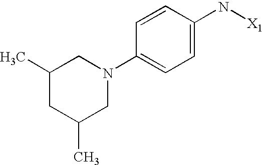 | C26H35N5O | 433.60 | 434 |
| 32 | 1-[1-(2-Di- methylaminoethyl)-1H-in- dol-5-yl]-3-[4-(4-phe- nylpiperidin-1-yl)phe- nyl]urea | 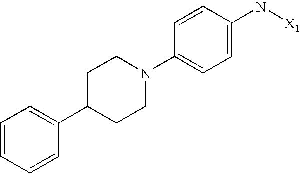 | C30H35N5O | 481.65 | 482 |
| 33 | 1-[1-(2-Di- methylaminoethyl)-1H-in- dol-5-yl]-3-[4-(4-methyl- piperidin-1-yl)phe- nyl]urea | 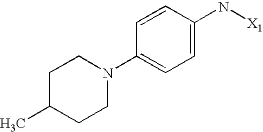 | C25H33N5O | 419.57 | 420 |
| 34 | 1-(4-Azepan-1-ylphe- nyl)-3-[1-(2-di- methylaminoethyl)-1H-in- dol-5-yl]urea | 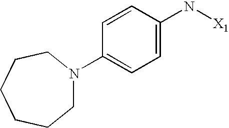 | C25H33N5O | 419.57 | 420 |
| 35 | 1-[4-(Benzyl- methylamino)phe- nyl]-3-[1-(2-di- methylaminoethyl)-1H-in- dol-5-yl]urea | 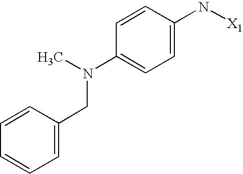 | C27H31N5O | 441.58 | 442 |
| 36 | 1-[1-(2-Dimethyl- aminoethyl)-1H-indol-5-yl]-3[4-(methyl- phenethylamino)-phe- nyl]urea | 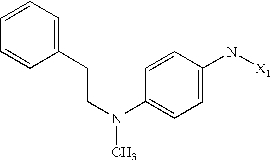 | C28H33N5O | 455.61 | 456 |
| 37 | 1-[4-(Butylmethyl- amino)phenyl]-3-[1-(2-di- methylaminoethyl)-1H-in- dol-5-yl]urea | 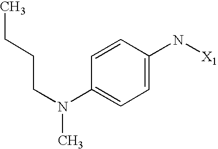 | C24H33N5O | 407.56 | 408 |
| 38 | 1-[4-(Benzylbutyl- amino)phenyl]-3-[1-(2-di- methylaminoethyl)-1H-in- dol-5-yl]urea | 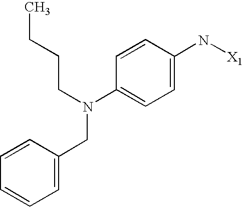 | C30H37N5O | 483.66 | 484 |
| 39 | 1-(4-Di- butylaminophenyl)-3-[1-(2-di- methylaminoethyl)-1H-in- dol-5-yl]urea | 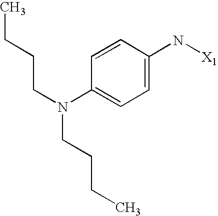 | C27H39N5O | 449.64 | 450 |
| 40 | 1-[1-(2-Dimethyl- aminoethyl)-1H-in- dol-5-yl]-3-[(4aR,8aS)-4-(octa- hydroisoquinolin-2-yl)phenyl]urea | 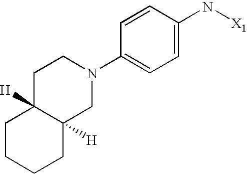 | C28H37N5O | 459.64 | 460 |
| 41 | 1-[1-(2-Di- methylaminoethyl)-1H-in- dol-5-yl]-3-[4-(2-methyl- pyrrolidin-1-yl)phe- nyl]urea | 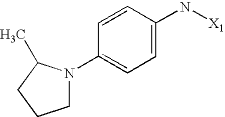 | C24H31N5O | 405.55 | 406 |
| 42 | 1-[1-(2-Di- methylaminoethyl)-1H-in- dol-5-yl]-3-[4-(5-eth- yl-2-methyl- piperidin-1-yl)phe- nyl]urea | 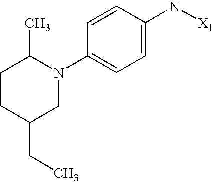 | C27H37N5O | 447.63 | 448 |
| 43 | 1-[1-(2-Dimethyl- aminoethyl)-1H-indol-5-yl]-3-[4-(methyl- pyridin-3-ylmethyl- amino)phenyl]urea | 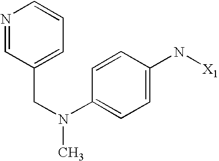 | C26H30N6O | 442.57 | 443 |
| 44 | 1-[4-(3-Aza- bicyclo[3.2.2]non-3-yl)phe- nyl]-3-[1-(2-di- methylaminoethyl)-1H-in- dol-5-yl]urea | 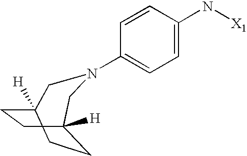 | C27H35N5O | 445.61 | 446 |
| 45 | 1-[1-(2-Di- methylaminoethyl)-1H-in- dol-5-yl]-3-[4-(2-iso- propylpyrrolidin-1-yl)phe- nyl]urea | 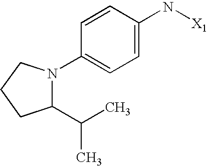 | C26H35N5O | 433.60 | 434 |
| 46 | 1-[1-(2-Di- methylaminoethyl)-1H-in- dol-5-yl]-3-[4-(2-iso- butylpyrrolidin-1-yl)phe- nyl]urea | 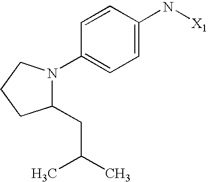 | C27H37N5O | 447.63 | 448 |
| 47 | 1-[1-(2-Di- methylaminoethyl)-1H-in- dol-5-yl]-3-[4-(3-phenyl- pyrrolidin-1-yl)phe- nyl]urea | 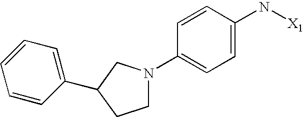 | C29H33N5O | 467.62 | 468 |
| 48 | 1-[1-(2-Dimethyl- aminoethyl)-1H-indol-5-yl]-3-[4-(3-tri- fluoromethyl- piperidin-1-yl)-phe- nyl]urea | 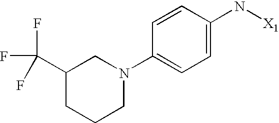 | C25H30F3N5O | 473.55 | 474 |
| 49 | 1-[1-(2-Dimethyl- aminoethyl)-1H-indol-5-yl]-3-[(4aR,8aR)-4-(octa- hydroisoquinolin-2-yl)phe- nyl]urea | 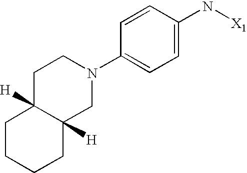 | C28H37N5O | 459.64 | 460 |
| 50 | 1-[4-(3,4-Dihydro-1H-iso- quinolin-2-yl)-phe- nyl]-3-[1-(2-di- methylaminoethyl)-1H-in- dol-5-yl]urea | 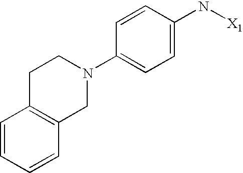 | C28H31N5O | 453.59 | 454 |
| 51 | 1-[1-(2-Di- methylaminoethyl)-1H-in- dol-5-yl]-3-[4-((1S,5R)-1,3,3-tri- methyl-6-aza- bicyclo[3.2.1]oct-6-yl)phe- nyl]urea | 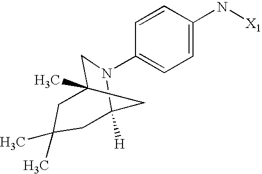 | C29H39N5O | 473.67 | 474 |
| 52 | 1-[1-(2-Di- methylaminoethyl)-1H-in- dol-5-yl]-3-(4-iso- butoxy-2,6-di- methylphenyl)urea | 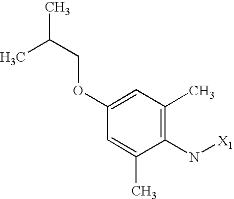 | C25H34N4O2 | 422.58 | 423 |
| 53 | 1-[1-(2-Di- methylaminoethyl)-1H-in- dol-5-yl]-3-(4-iso- butoxy-3-methoxy- phenyl)urea | 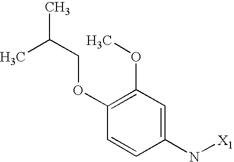 | C24H32N4O3 | 424.55 | 425 |
| 54 | 1-[1-(2-Di- methylaminoethyl)-1H-in- dol-5-yl]-3-(4-iso- butoxy-2-methyl- phenyl)urea | 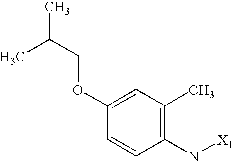 | C24H32N4O2 | 408.55 | 409 |
| 55 | 1-[1-(2-Di- methylaminoethyl)-1H-in- dol-5-yl]-3-(4-iso- butoxy-2,5-di- methylphenyl)urea | 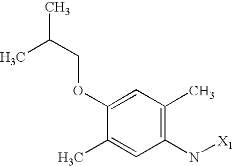 | C25H34N4O2 | 422.58 | 423 |
| 56 | 1-(3,5-Dichloro-4-iso- butoxyphenyl)-3-[1-(2-di- methylaminoethyl)-1H-in- dol-5-yl]urea | 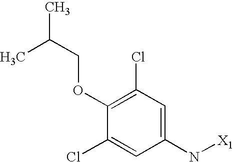 | C23H28Cl2N4C | 463.41 | 463 |
| 57 | 1-[1-(2-Dimethyl- aminoethyl)-1H-in- dol-5-yl]-3-(4-iso- butoxy-3-nitro- phenyl)urea | 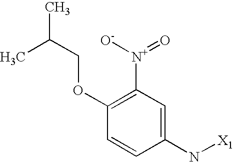 | C23H29N5O4 | 439.52 | 440 |
| 58 | 1-[1-(2-Dimethyl- aminoethyl)-1H-in- dol-5-yl]-3-(4-iso- butoxy-3-methyl- phenyl)urea | 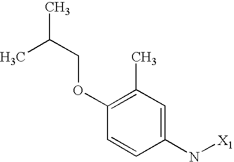 | C24H32N4O2 | 408.55 | 409 |
| 59 | 1-Benzyl-3-[1-(2-di- methylaminoethyl)-1H-in- dol-5-yl]-1-(4-iso- butoxyphenyl)urea | 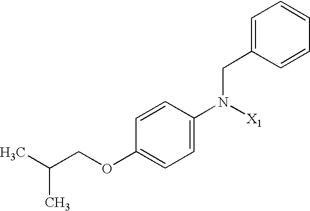 | C30H36N4O2 | 484.65 | 485 |
| 60 | 1-(3-Chloro-4-iso- butoxy-5-methyl- phenyl)-3-[1-(2-di- methylaminoethyl)-1H-in- dol-5-yl]urea | 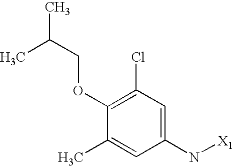 | C24H31ClN4O2 | 442.99 | 443 |
| 61 | 1-[1-(2-Dimethyl- aminoethyl)-1H-in- dol-5-yl]-3-(4-iso- butoxy-2-nitro- phenyl)urea | 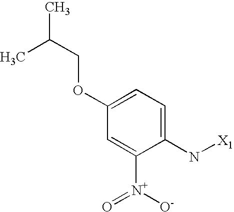 | C23H29N5O4 | 439.52 | 440 |
| 62 | 1-[1-(2-Dimethyl- aminoethyl)-1H-in- dol-5-yl]-3-(4-iso- butoxy-2,3-di- methylphenyl)urea | 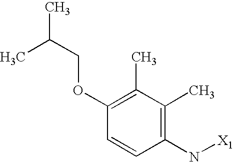 | C25H34N4O2 | 422.58 | 423 |
| 63 | 1-[1-(2-Dimethyl- aminoethyl)-1H-in- dol-5-yl]-3-(2-fluoro-4-iso- butoxyphenyl)urea | 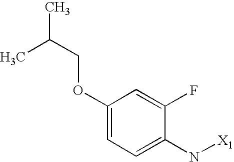 | C23H29FN4O2 | 412.51 | 413 |
| 64 | 1-(3-Chloro-4-iso- butoxyphenyl)-3-[1-(2-di- methylaminoethyl)-1H-in- dol-5-yl]urea | 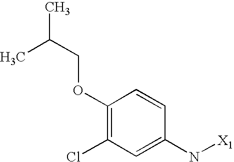 | C23H29ClN4O2 | 428.97 | 429 |
| 65 | 1-[1-(2-Dimethyl- aminoethyl)-1H-in- dol-5-yl]-3-(3-fluoro-4-iso- butoxyphenyl)urea | 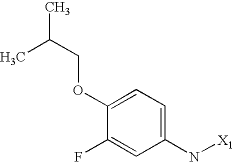 | C23H29FN4O2 | 412.51 | 413 |
| 66 | 1-(2-Chloro-4-iso- butoxyphenyl)-3-[1-(2-di- methylaminoethyl)-1H-in- dol-5-yl]urea | 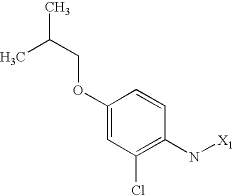 | C23H29ClN4O2 | 428.97 | 429 |
| 67 | Methyl 5-{3-[1-(2-di- methylaminoethyl)-1H-in- dol-5-yl]ureido}-2-iso- butoxy benzoate | 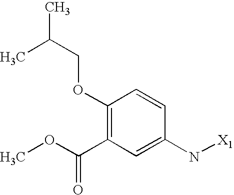 | C25H32N4O4 | 452.56 | 453 |
| 68 | 1-(3-Cyano-4-iso- butoxyphenyl)-3-[1-(2-di- methylaminoethyl)-1H-in- dol-5-yl]urea | 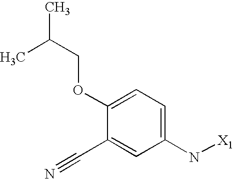 | C24H29N5O2 | 419.53 | 420 |
| 69 | 1-[1-(2-Dimethyl- aminoethyl)-1H-in- dol-5-yl]-3-(4-iso- butoxy-3,5-di- methylphenyl)urea | 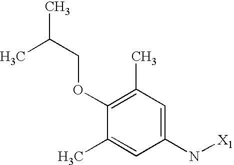 | C25H34N4O2 | 422.58 | 423 |
| 70 | 1-[1-(2-Dimethyl- aminoethyl)-1H-in- dol-5-yl]-3-(4-iso- butoxy-2-tri- fuoromethylphenyl)urea | 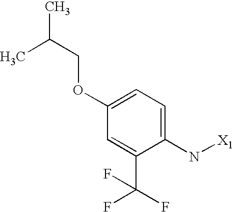 | C24H29F3N4O | 462.52 | 463 |
| 71 | 1-(4-Butylphenyl)-3-[1-(2-di- methylaminoethyl)-1H-in- dol-5-yl]urea | 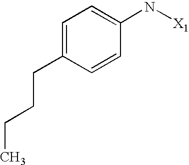 | C23H30N4O | 378.52 | 379 |
| 72 | 1-[1-(2-Dimethyl- aminoethyl)-1H-in- dol-5-yl]-3-(4-iso- butoxyphenyl)urea | 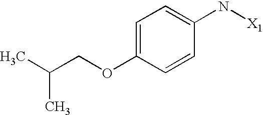 | C23H30N4O2 | 394.52 | 395 |
| 73 | 1-[1-(2-Dimethyl- aminoethyl)-1H-in- dol-5-yl]-3-[4-(py- ridin-3-yloxy)phe- nyl]urea | 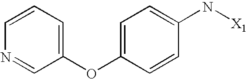 | C24H25N5O2 | 415.50 | 416 |
| 74 | 1-(3-Cyclopentyloxy-4-methoxy- phenyl)-3-[1-(2-di- methylaminoethyl)-1H-in- dol-5-yl]urea | 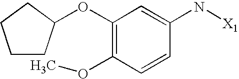 | C25H32N4O3 | 436.56 | 437 |
| 75 | 1-(4-Benzenesulfonyl-2-nitro- phenyl)-3-[1-(2-dimethyl- aminoethyl)-1H-in- dol-5-yl]urea | 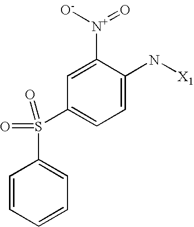 | C25H25N5O5S | 507.57 | 508 |
| 76 | 1-[1-(2-Dimethyl- aminoethyl-1H-in- dol-5-yl]-3-[4-(2-methoxy- phenoxy)-phenyl]urea | 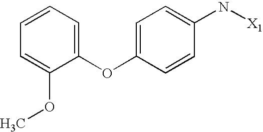 | C26H28N4O3 | 444.54 | 445 |
| 77 | 1-[4-(3-Chloro- phenoxy)-phe- nyl]-3-[1-(2-dimethyl- aminoethyl)-1H-in- dol-5-yl]urea | 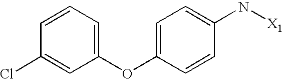 | C25H25ClN4O2 | 448.96 | 449 |
| 78 | 1-Biphenyl-4-yl-3-[1-(2-di- methylaminoethyl)-1H-in- dol-5-yl]urea | 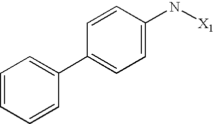 | C25H26N4O | 398.51 | 399 |
| 79 | 1-[1-(2-Dimethyl- aminoethyl)-1H-indol-5-yl]-3-(2-meth- oxy-4-phenyl- aminophenyl)-urea | 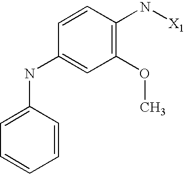 | C26H29N5O2 | 443.55 | 444 |
| 80 | 1-(4-Benzyloxyphenyl)-3-[1-(2-di- methylaminoethyl)-1H-in- dol-5-yl]urea | 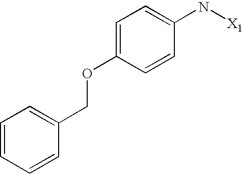 | C26H28N4O2 | 428.54 | 429 |
| 81 | 1-[1-(2-Di- methylaminoethyl)-1H-in- dol-5-yl]-3-(4′-fluoro- biphenyl-4-yl)urea | 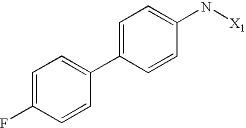 | C25H25FN4O | 416.50 | 417 |
| 82 | 1-(4-Benzylphenyl)-3-[1-(2-di- methylaminoethyl)-1H-in- dol-5-yl]urea | 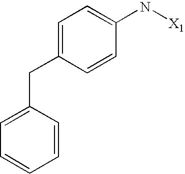 | C26H28N4O | 412.54 | 413 |
| 83 | 1-[1-(2-Di- methylaminoethyl)-1H-in- dol-5-yl]-3-(4-py- ridin-4-ylmeth- ylphenyl)urea | 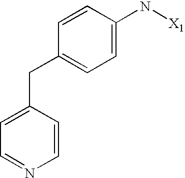 | C25H27N5O | 413.53 | 414 |
| 84 | 1-[1-(2-Di- methylaminoethyl)-1H-in- dol-5-yl]-3-(4-p-tolyl- oxyphenyl)urea | 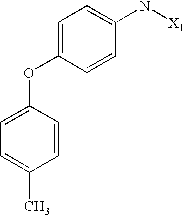 | C26H28N4O2 | 428.54 | 429 |
| 85 | 1-[1-(2-Di- methylaminoethyl)-1H-in- dol-5-yl]-3-(4-phenyl- sulfanylphenyl)urea | 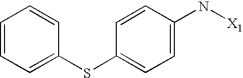 | C25H26N4OS | 430.58 | 431 |
| 86 | 1-[1-(2-Di- methylaminoethyl)-1H-in- dol-5-yl]-3-[4-(3-tri- fluoromethyl- phenoxy)phenyl]urea | 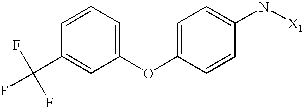 | C26H25F3N4O | 482.51 | 483 |
| 87 | 1-(4-Butyl-2-methyl- phenyl)-3-[1-(2-di- methylaminoethyl)-1H-in- dol-5-yl]urea | 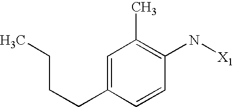 | C24H32N4O | 392.55 | 393 |
| 88 | 1-(4′-Cyanobiphenyl-4-yl)-3-[1-(2-di- methylaminoethyl)-1H-in- dol-5-yl]urea | 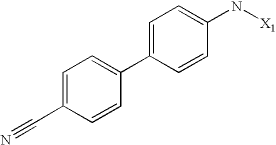 | C26H25N5O | 423.52 | 424 |
| 89 | 1-[4-(4-Chloro- phenoxy)-2-tri- fluoromethylphenyl]-3-[1-(2-di- methylaminoethyl)-1H-in- dol-5-yl]urea | 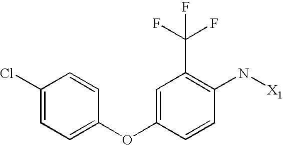 | C26H24ClF3N4 | 516.95 | 517 |
| 90 | 1-[3-Chloro-4-(py- rimidin-2-yloxy)phe- nyl]-3-[1-(2-di- methylaminoethyl)-1H-in- dol-5-yl]urea | 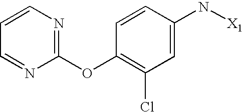 | C23H23ClN6O2 | 450.93 | 451 |
| 91 | 1-[1-(2-Di- methylaminoethyl)-1H-in- dol-5-yl]-3-(5-meth- oxy-2-meth- ylbiphenyl-4-yl)urea | 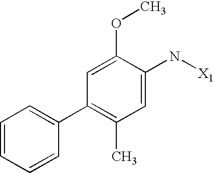 | C27H30N4O2 | 442.57 | 443 |
| 92 | 1-[1-(2-Di- methylaminoethyl)-1H-in- dol-5-yl]-3-[4-(pipe- ridine-1-sulfo- nyl)phenyl]urea | 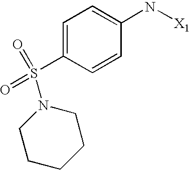 | C24H31N5O3S | 469.61 | 470 |
| 93 | Ethyl 5-(4-{3-[1-(2-di- methylaminoethyl)-1H-in- dol-5-yl]urei- do}phenyl)-2-methyl- furan-3-carboxylate | 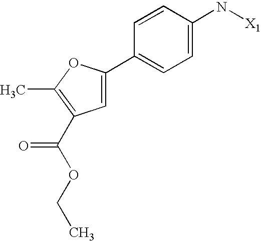 | C27H30N4O4 | 474.56 | 475 |
| 94 | 1-(4-Benzooxazol-2-ylphe- nyl)-3-[1-(2-di- methylaminoethyl)-1H-in- dol-5-yl]urea | 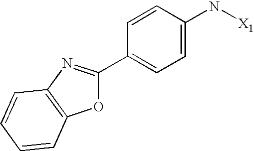 | C26H25N5O2 | 439.52 | 440 |
| 95 | 1-[1-(2-Di- methylaminoethyl)-1H-in- dol-5-yl]-3-[4-(pipe- ridine-1-carbo- nyl)phenyl]urea | 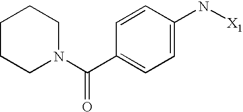 | C25H31N5O2 | 433.56 | 434 |
| 96 | 1-[3-Cyano-4-(3-tri- fluoromethylphenyl- sulfanyl)phenyl]-3-[1-(2-di- methylamino-ethyl)-1H-in- dol-5-yl]urea | 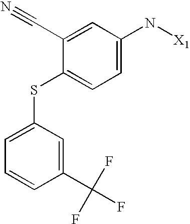 | C27H24F3N5O | 523.58 | 524 |
| 97 | 1-[1-(2-Di- methylaminoethyl)-1H-in- dol-5-yl]-3-(4-hepta- fluoropropyl-sulfanyl- phenyl)urea | 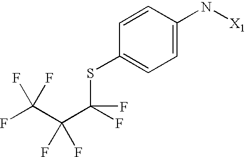 | C22H21F7N4O | 522.49 | 523 |
| 98 | 1-(4-Benzenesulfonyl-3-chloro- phenyl)-3-[1-(2-di- methylaminoethyl)-1H-in- dol-5-yl]urea | 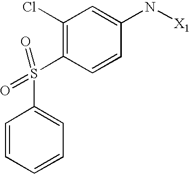 | C25H25ClN4O3 | 497.02 | 497 |
| 99 | 1-[1-(2-Di- methylaminoethyl)-1H-in- dol-5-yl]-3-[4-(py- rimidin-2-yloxy)phe- nyl]urea | 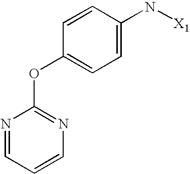 | C23H24N6O2 | 416.49 | 417 |
| 100 | 1-[1-(2-Di- methylaminoethyl)-1H-in- dol-5-yl]-3-(2-methoxy- biphenyl-4-yl)urea | 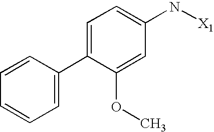 | C26H28N4O2 | 428.54 | 429 |
| 101 | 1-[1-(2-Di- methylaminoethyl)-1H-in- dol-5-yl]-3-(6-methoxy- biphenyl-3-yl)urea | 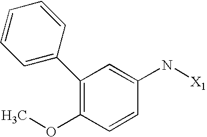 | C26H28N4O2 | 428.54 | 429 |
| 102 | 1-[1-(2-Di- methylaminoethyl)-1H-in- dol-5-yl]-3-(4-[1,3]dithio- lan-2-ylphenyl)urea | 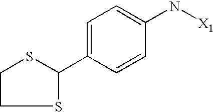 | C22H26N4OS2 | 426.61 | 427 |
| 103 | 1-[1-(2-Dimethyl- aminoethyl)-1H-in- dol-5-yl]-3-[4-(thio- phen-2-ylsulfa- nyl)phenyl]urea | 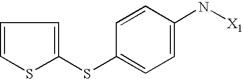 | C23H24N4OS2 | 436.60 | 437 |
| 104 | 3-[1-(2-Di- methylaminoethyl)-1H-in- dol-5-yl]-1-(4-methoxy- phenyl)-1-methylurea | 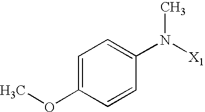 | C21H26N4O2 | 366.47 | 367 |
| 105 | 1-[4-(2-Chloro- phenoxy)-phenyl]-3-[1-(2-di- methylaminoethyl)-1H-in- dol-5-yl]urea | 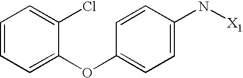 | C25H25ClN4O2 | 448.96 | 449 |
| 106 | 1-[1-(2-Di- methylaminoethyl)-1H-in- dol-5-yl]-3-(6-phenoxy- pyridin-3-yl)urea | 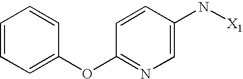 | C24H25N5O2 | 415.50 | 416 |
| 107 | 1-[1-(2-Di- methylaminoethyl)-1H-in- dol-5-yl]-3-(4-m-tolyl- oxyphenyl)urea | 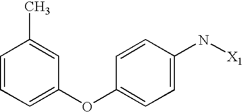 | C26H28N4O2 | 428.54 | 429 |
| 108 | 1-[1-(2-Di- methylaminoethyl)-1H-in- dol-5-yl]-3-(4-o-tolyl- oxyphenyl)urea | 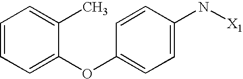 | C26H28N4O2 | 428.54 | 429 |
| 109 | 1-[1-(2-Dimethyl- aminoethyl)-1H-indol-5-yl]-3-[4-(3-meth- oxyphenoxy)-phenyl]urea | 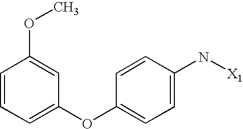 | C26H28N4O3 | 444.54 | 445 |

The molecule ion peak ([M+H]⁺) was taken from ESI mass spectra.

The examples 20-51 and 71-109 were prepared according to Example 1.

### Synthesis of Examples 52-70

Carbonyldiimidazole (0.25 mmol) was added to 1-(2-dimethylaminoethyl)-1H-indol-5-ylamine (0.25 mmol) in dimethylformamide (1 mL) at 0° C. After 1 hour at room temperature, the reaction solution was cooled again to 0° C. and the appropriate aminophenol (0.25 mmol) was added. After 15 hours at room temperature, cesium carbonate (0.5 mmol) and isobutyl iodide (0.5 mmol) were added and the solution was heated at 80° C. for 2 hours. The reaction solutions were filtered and the filtrate was washed with sodium bicarbonate (5% strength) and sodium chloride solution (5% strength). The organic phase was dried and concentrated. The crude product was purified by preparative HPLC. Thus, the product having the molecular weight indicated in Table 3 and the molecule ion peak of the mass spectrum, likewise indicated in Table 3, was obtained.

### Precursors of Examples 20-51

A mixture of 4-fluoronitrobenzene (0.35 mmol), potassium carbonate (0.7 mmol), the appropriate amine and dimethylformamide (1 mL) was heated to 100° C. for three hours. The reaction solution was filtered and washed with sodium chloride solution (5% strength). The organic phase was dried and concentrated. The 4-nitroaniline obtained as crude product was dissolved in glacial acetic acid (1 mL) and zinc dust (0.25 g) was added. After a reaction time of 3 hours, the reaction solution was diluted with ethyl acetate (10 mL), filtered and the filtrate was washed with sodium chloride solution (5% strength). The filtrate was dried and concentrated. The obtained crude product, 4-substituted aniline, was reacted further without any further purification.

The following 4-nitroanilines were prepared:

1-(4-nitrophenyl)azocan

cyclohexylmethyl-(4-nitrophenyl)amine

1-(4-nitrophenyl)pyrrolidine

2,5-dimethyl-1-(4-nitrophenyl)pyrrolidine

1-(4-nitrophenyl)-1,2,3,6-tetrahydropyridine

2,6-dimethyl-4-(4-nitrophenyl)morpholine

4-(4-nitrophenyl)thiomorpholine

2-methyl-1-(4-nitrophenyl)piperidine

2-ethyl-1-(4-nitrophenyl)piperidine

3-methyl-1-(4-nitrophenyl)piperidine

3,3-dimethyl-1-(4-nitrophenyl)piperidine

3,5-dimethyl-1-(4-nitrophenyl)piperidine

1-(4-nitrophenyl)-4-phenylpiperidine

4-methyl-1-(4-nitrophenyl)piperidine

2-(4-nitrophenyl)-1,2,3,4-tetrahydroisoquinoline

1-(4-nitrophenyl)azepan

benzylmethyl-(4-nitrophenyl)amine

methyl-(4-nitrophenyl)phenethylamine

butylmethyl-(4-nitrophenyl)amine

benzylbutyl-(4-nitrophenyl)amine

dibutyl-(4-nitrophenyl)amine

(4aR,8aS)-2-(4-nitrophenyl)decahydroisoquinoline

2-methyl-1-(4-nitrophenyl)pyrrolidine

5-ethyl-2-methyl-1-(4-nitrophenyl)piperidine

methyl-(4-nitrophenyl)pyridine-3-ylmethylamine

3-(4-nitrophenyl)-3-azabicyclo[3.2.2]nonane

2-isopropyl-1-(4-nitrophenyl)pyrrolidine

2-isobutyl-1-(4-nitrophenyl)pyrrolidine

1-(4-nitrophenyl)-3-phenylpyrrolidine

1-(4-nitrophenyl)-3-trifluoromethylpiperidine

(4aR,8aR)-2-(4-nitrophenyl)dekahydroisoquinoline

(1S,5R)-1,3,3-trimethyl-6-(4-nitrophenyl)-6-azabicyclo[3.2.1]octane

All of the 4-nitroanilines listed above showed the expected molecule ion peak in the ESI mass spectrum.

The following 4-substituted anilines were prepared:

4-azocan-1-ylphenylamine

N-cyclohexyl-N-methylbenzene-1,4-diamine

4-pyrrolidin-1-ylphenylamine

4-(2,5-dimethylpyrrolidin-1-yl)phenylamine

4-(3,6-dihydro-2H-pyridin-1-yl)phenylamine

4-(2,6-dimethylmorpholin-4-yl)phenylamine

4-thiomorpholin-4-ylphenylamine

4-(2-methylpiperidin-1-yl)phenylamine

4-(2-ethylpiperidin-1-yl)phenylamine

4-(3-methylpiperidin-1-yl)phenylamine

4-(3,3-dimethylpiperidin-1-yl)phenylamine

4-(3,5-dimethylpiperidin-1-yl)phenylamine

4-(4-phenylpiperidin-1-yl)phenylamine

4-(4-methylpiperidin-1-yl)phenylamine

4-(3,4-dihydro-1H-isoquinolin-2-yl)phenylamine

4-azepan-1-ylphenylamine

N-benzyl-N-methylbenzene-1,4-diamine

N-methyl-N-phenethylbenzene-1,4-diamine

N-butyl-N-methylbenzene-1,4-diamine

N-benzyl-N-butylbenzene-1,4-diamine

N,N-dibutylbenzene-1,4-diamine

(4aR,8aS)4-(octahydroisoquinolin-2-yl)phenylamine

4-(2-methylpyrrolidin-1-yl)phenylamine

4-(5-ethyl-2-methylpiperidin-1-yl)phenylamine

N-methyl-N-pyridin-3-ylmethylbenzene-1,4-diamine

4-((1S,5R)-1,3,3-trimethyl-6-azabicyclo[3.2.1]oct-6-yl)phenylamine

4-(3-azabicyclo[3.2.2]non-3-yl)phenylamine

4-(2-isopropylpyrrolidin-1-yl)phenylamine

4-(2-isobutylpyrrolidin-1-yl)phenylamine

4-(3-phenylpyrrolidin-1-yl)phenylamine

4-(3-trifluoromethylpiperidin-1-yl)phenylamine

(4aR,8aR)4-(octahydroisoquinolin-2-yl)phenylamine.

All of the 4-substituted anilines listed above showed the expected molecule ion peak in the ESI mass spectrum.

### Example 110

4-Phenoxyphenyl[1-(2-dimethylaminoethyl)-1H-indol-5-yl]carbamate

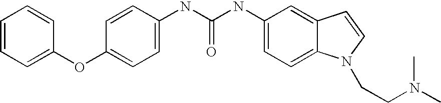

The compound was prepared according to Example 1 by reacting the carbonyldiimidazole-activated indolamine with deprotonated 4-phenoxyphenol. Thus, the product having a molecular weight of 415.50 (C₂₅H₂₅N₃O₃); MS (ESI): 416 (M+H⁺) was obtained.

### Example 111

1-(2-Imidazol-1-ylmethyl-1-methyl-1H-indol-5-yl)-3-(4-phenoxyphenyl)urea

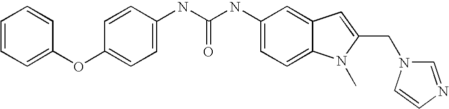

Mesyl chloride (47 μl) was added to 1-(2-hydroxymethyl-1-methyl-1H-indol-5-yl)-3-(4-phenoxyphenyl)urea (0.2 g) and triethylamine (0.16 mL) in dichloromethane (4 mL) at 0° C. After 10 minutes, imidazole (185 mg) was added. After 12 hours, the reaction solution was washed with sodium chloride solution, dried and concentrated. The crude product was purified by preparative HPLC. Thus, the product having a molecular weight of 437.51 (C₂₆H₂₃N₅O₂); MS (ESI): 438 (M+H⁺) was obtained.

1-(2-Hydroxymethyl-1-methyl-1H-indol-5-yl)-3-(4-phenoxyphenyl)urea

(5-Amino-1-methyl-1H-indol-2-yl)methanol was reacted with 4-phenoxyaniline and carbonyldiimidazole, as described in Example 1. Thus, the product having a molecular weight of 387.44 (C₂₃H₂₁N₃O₃); MS (ESI): 388 (M+H⁺) was obtained.

(5-Amino-1-methyl-1H-indol-2-yl)methanol

(1-Methyl-5-nitro-1H-indol-2-yl)methanol was hydrogenated as described in Example 3. Thus the product having a molecular weight of 176.22 (C₁₀H₁₂N₂O); MS (ESI): 177 (M+H⁺) was obtained.

### Example 112

1-[1-Methyl-2-(2-methyl-4,5-dihydroimidazol-1-ylmethyl)-1H-indol-5-yl]-3-(4-phenoxyphenyl)urea

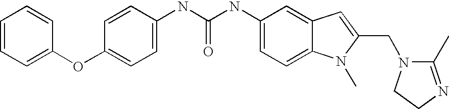

The compound was prepared from 1-(2-hydroxymethyl-1-methyl-1H-indol-5-yl)-3-(4-phenoxyphenyl)urea and 2-methyl-4,5-dihydroimidazole, as described in Example 111. Thus, the product having a molecular weight of 453,55 (C₂₇H₂₇N₅O₂); MS (ESI): 454 (M+H⁺) was obtained.

### Example 113

1-(2-Cyclohexylaminomethyl-1-methyl-1H-indol-5-yl)-3-(4-phenoxyphenyl)urea

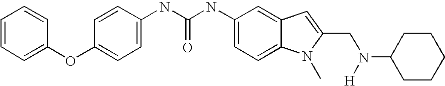

The compound was prepared from 1-(2-hydroxymethyl-1-methyl-1H-indol-5-yl)-3-(4-phenoxyphenyl)urea and cyclohexylamine, as described in Example 111. Thus, the product having a molecular weight of 468.60 (C₂₉H₃₂N₄O₂); MS (ESI): 469 (M+H⁺) was obtained.

### Example 114

1-[2-(3-Dimethylaminopyrrolidin-1-ylmethyl)-1-methyl-1H-indol-5-yl]-3-(4-phenoxyphenyl)urea

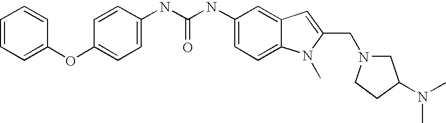

The compound was prepared from 1-(2-hydroxymethyl-1-methyl-1H-indol-5-yl)-3-(4-phenoxyphenyl)urea and 3-dimethylaminopyrrolidine, as described in Example 111. Thus, the product having a molecular weight of 483.62 (C₂₉H₃₃N₅O₂); MS (ESI): 484 (M+H⁺) was obtained.

### Example 115

1-[2-(4-Hydroxypiperidin-1-ylmethyl)-1-methyl-1H-indol-5-yl]-3-(4-phenoxyphenyl)urea

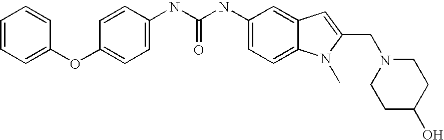

The compound was prepared from 1-(2-hydroxymethyl-1-methyl-1H-indol-5-yl)-3-(4-phenoxyphenyl)urea and 4-hydroxypiperidin, as described in Example 111. Thus, the product having a molecular weight of 470.58 (C₂₈H₃₀N₄O₃); MS (ESI): 471 (M+H⁺) was obtained.

### Example 116

1-[1-Methyl-2-(4-phenylpiperidin-1-ylmethyl)-1H-indol-5-yl]-3-(4-phenoxyphenyl)urea

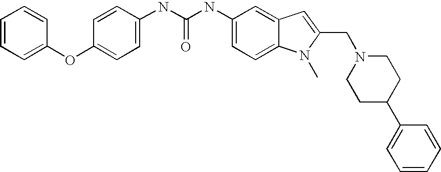

The compound was prepared from 1-(2-hydroxymethyl-1-methyl-1H-indol-5-yl)-3-(4-phenoxyphenyl)urea and 4-phenylpiperidine, as described in Example 111. Thus, the product having a molecular weight of 530.68 (C34H₃₄N₄O₂); MS (ESI): 531 (M+H⁺) was obtained.

### Example 117

N-(1-{1-Methyl-5-[3-(4-phenoxyphenyl)ureido]-1H-indol-2-ylmethyl}pyrrolidin-3-yl)acetamide

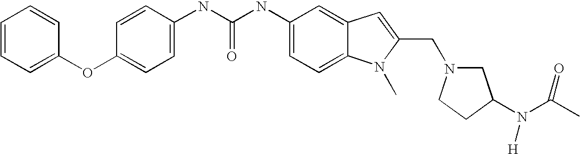

The compound was prepared from 1-(2-hydroxymethyl-1-methyl-1H-indol-5-yl)-3-(4-phenoxyphenyl)urea and pyrrolidin-3-ylacetamide, as described in Example 111. Thus, the product having a molecular weight of 497.60 (C₂₉H₃₁N₅O₃); MS (ESI): 498 (M+H⁺) was obtained.

### Example 118

1-(4-Phenoxyphenyl)-3-(2-pyrrolidin-1-ylmethylbenzofuran-5-yl)urea

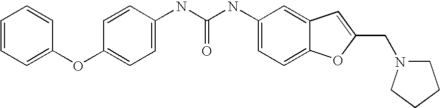

The compound was prepared from 2-pyrrolidin-1-ylmethylbenzofuran-5-ylamine and 4-phenoxyaniline, as described in Example 1. Thus, the product having a molecular weight of 427.51 (C₂₆H₂₅N₃O₃); MS (ESI): 428 (M+H⁺) was obtained.

2-Pyrrolidin-1-ylmethylbenzofuran-5-ylamine

The compound was prepared by hydrogenation of 1-(5-nitrobenzofuran-2-ylmethyl)pyrrolidine, as described in Example 3. Thus, the product having a molecular weight of 216.29 (C₁₃H₁₆N₂O); MS (ESI): 217 (M+H⁺) was obtained.

1-(5-Nitrobenzofuran-2-ylmethyl)pyrrolidine

The compound was prepared from (5-nitrobenzofuran-2-yl)methanol, as described in Example 3. Thus, the product having a molecular weight of 246.27 (C₁₃H₁₄N₂O₃); MS (ESI): 247 (M+H⁺) was obtained.

(5-Nitrobenzofuran-2-yl)methanol

The compound was prepared by reduction of methyl 5-nitrobenzofuran 2-carboxylate, as described in Example 3. Thus, the product having a molecular weight of 193.16 (C₉H₇NO₄); MS (ESI): 194 (M+H⁺) was obtained.

### Example 119

1-(4-Phenoxyphenyl)-3-(2-pyrrolidin-1-ylmethylbenzo[b]thiophen-5-yl)urea

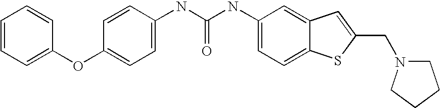

The compound was prepared from 2-pyrrolidin-1-ylmethylbenzo[b]thiophen-5-ylamine and 4-phenoxyaniline, as described in Example 1. Thus, the product having a molecular weight of 443.57 (C₂₆H₂₅N₃O₂S); MS (ESI): 444 (M+H⁺) was obtained.

2-Pyrrolidin-1-ylmethylbenzo[b]thiophen-5-ylamine

The compound was prepared by hydrogenation of 1-(5-nitrobenzo[b]thiophen-2-ylmethyl)pyrrolidine, as described in Example 3. Thus, the product having a molecular weight of 232.35 (C₁₃H₁₆N₂S); MS (ESI): 233 (M+H⁺) was obtained.

1-(5-Nitrobenzo[b]thiophen-2-ylmethyl)pyrrolidine

The compound was prepared from (5-nitrobenzo[b]thiophen-2-yl)methanol, as described in Example 3. Thus, the product having a molecular weight of 262.33 (C₁₃H₁₄N₂O₂S); MS (ESI): 263 (M+H⁺) was obtained.

(5-Nitrobenzo[b]thiophen-2-yl)methanol

The compound was prepared by reduction of methyl 5-nitrobenzo[b]thiophene 2-carboxylate, as described in Example 3. Thus, the product having a molecular weight of 209.23 (C₉H₇NO₃S); MS (ESI): 210 (M+H⁺) was obtained.

In general, all of the basic compounds described were obtained either as free bases or in the form of a salt of one of the following acids: formic acid, trifluoroacetic acid or hydrochloric acid.

1. A compound of formula I, 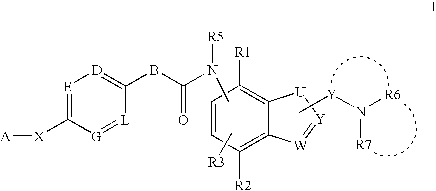
wherein
A is (C₁-C₈)alkyl, (C₀-C₈)alkylenearyl; a 3- to 12-membered mono- or bicyclic ring which may contain one or more heteroatoms selected from the group consisting of N, O, and S, and the 3- to 12-membered ring may carry further substituents selected from the group consisting of F, Cl, Br, NO₂, CF₃, OCF₃, CN, (C₁-C₆)alkyl, aryl, CON(R37)(R38), N(R39)(R40), OH, O—(C₁-C₆)alkyl, S—(C₁-C₆)alkyl, and NHCO(C₁-C₆)alkyl;
X is a bond, C(R8)(R9), C(OR10)(R11), O, N(R12), S, SO, SO₂, or CO; R8, R9, R10, R11, R12 are, independently of one another, H or (C₁-C₆)alkyl;
D is C(R41);
E is C(R42);
G is C(R43);
L is C(R44);
R1, R2, R3, R41, R42, R43, R44 are, independently of one another, H, F, Cl, Br, J, OH, CF₃, NO₂, CN, OCF₃, O—(C₁-C₆)alkyl, (C₁-C₄)alkoxyalkyl, S—(C₁-C₆)alkyl, (C₁-C₆)alkyl, (C₂-C₆)alkenyl, (C₃-C₈)cycloalkyl, O—(C₃-C₈)cycloalkyl, O—(C₃-C₈)cycloalkenyl, O—(C₃-C₈)-cycloalkenyl, (C2-C₆)alkynyl, (C₀-C₈)alkylenearyl, —O—(C₀-C₈)alkylenearyl, S—aryl, N(R13)(R14), SO₂-CH₃, COOH, COO—(C₁-C₆)alkyl, CON(R15)(R16), N(R17)CO(R18), N(R 19)SO₂(R20), CO(R21), or a 5- to 7-membered heterocycle having 1-4 heteroatoms;
R13, R14 are, independently of one another, H, (C₁-C₆)alkyl, or R13 and R14, together with the nitrogen atom to which they are bonded, form a 5- to 6-membered ring, wherein, in the case of the 6-membered ring, a CH₂ group may be replaced by O or S;
R15, R16 are, independently of one another, H, (C₁-C₆)alkyl, or R15 and R16, together with the nitrogen atom to which they are bonded, form a 5- to 6-membered ring, wherein, in the case of the 6-membered ring, a CH₂ group may be replaced by O or S;
R17, R19 are, independently of one another, H or (C₁-C₆)alkyl;
R18, R20, R21 are, independently of one another, (C₁-C₆)alkyl, or aryl;
B is N(R24) or O;
R24 is H or (C₁-C₆)alkyl;
R5 is H or (C₁-C₆)alkyl;
W is N;
T is C(R26);
R26 is H, (C₁-C₆)alkyl, aryl, (C₀-C₈)alkylenearyl, or a bond to Y;
U is N(R27);
R27, is H, (C₁-C₆)alkyl, or a bond to Y;
Y is (C₁-C₈)alkylene, in which one or more carbons may be replaced by O, S, SO, SO₂, C(R32)(R33), CO, C(R34)(OR35), or N(R36);
R32, R33, R34, R35, R36 are, independently of one another, H, (C₁-C₆)alkyl, or aryl;
R6, R7 are, independently of one another, H, (C₁-C₆)alkyl, (C₃-C₇)cycloalkyl, or R6 and Y or R6 and R7, together with the nitrogen atom to which they are bonded, form a 3- to 8-membered ring in which one or more carbons may be replaced by O, N, or S, and the 3- to 8-membered ring may carry further substituents such as (C₁-C₆)alkyl, aryl, CON(R37)(R38), N(R39)(R40), OH, O—(C₁-C₆)alkyl, or NHCO(C₁-C₆)alkyl;
R37, R38, R39, R40 are, independently of one another, H or (C₁-C₆)alkyl; and the physiologically acceptable salts thereof.

2. The compound of claim 1, wherein
A is (C₂-C₇)alkyl, (C₀-C₃)alkylenearyl; a 4- to 10-membered mono- or bicyclic ring which may contain one or more heteroatoms selected from the group consisting of N, O, and S, and the 4- to 10-membered ring may carry further substituents selected from the group consisting of F, Cl, Br, NO₂, CF₃, (C₁-C₆)alkyl, aryl, CON(R37)(R38), N(R39)(R40), O—(C₁-C₆)alkyl, and NHCO(C₁-C₆)alkyl;
X is a bond, C(R8)(R9), O, N(R12), S, or SO₂;
R8, R9, R12 are, independently of one another, H or (C₁-C₆)alkyl;
D is C(R41);
E is C(R42);
G is C(R43);
L is C(R44);
R1, R2, R3, R41, R42, R43, R44 are, independently of one another, H, F, Cl, Br, CF₃, NO₂, O—(C₁-C₆)alkyl, (C₁-C₆)alkyl, (C₃-C₈)cycloalkyl, O—(C₃-C₈)cycloalkyl, (C₂-C₆)alkynyl, (C₀-C₈)alkylenearyl, —O—(C₀-C₃)alkylenearyl, S-aryl, N(R13)(R14), SO₂-CH₃, COO—(C₁-C₆)alkyl, CON(R15)(R16), N(R17)CO(R18), N(R19)SO₂(R20), or CO(R21);
R13, R14 are, independently of one another, H, (C₁-C₆)alkyl, or R13 and R14, together with the nitrogen atom to which they are bonded, form a 5- to 6-membered ring, wherein, in the case of the 6-membered ring, a CH₂ group may be replaced by O or S;
R15, R16 are, independently of one another, H, (C₁-C₆)alkyl, or R15 and R16, together with the nitrogen atom to which they are bonded, form a 5- to 6-membered ring, wherein, in the case of the 6-membered ring, a CH₂ group may be replaced by O or S;
R17, R19 are, independently of one another, H, or (C₁-C₆)alkyl;
R18, R20, R21 are, independently of one another, (C₁-C₆)alkyl, or aryl;
B is N(R24) or O;
R24 is H or (C₁-C₆)alkyl;
R5 is H or (C₁-C₆)alkyl;
W is N;
T is C(R26);
R26 is H, (C₁-C₆)alkyl, aryl, or a bond to Y;
U is N(R27);
R27 is H, (C₁-C₆)alkyl, or a bond to Y;
Y is (C₁-C₄)alkylene, in which a carbon may be replaced by SO₂, C(R32)(R33), CO, or N(R36);
R32, R33, R36 are, independently of one another, H, (C₁-C₆)alkyl, or aryl;
R6, R7 are, independently of one another, H, (C₁-C₆)alkyl, (C₃-C₇)cycloalkyl, or R6 and Y or R6 and R7, together with the nitrogen atom to which they are bonded, form a 4- to 7-membered ring in which one or more carbons may be replaced by 0, N, or S, and the 4- to 7-membered ring may carry further substituents selected from the group consisting of (C₁-C₆)alkyl, aryl, CON(R37)(R38), N(R39)(R40), OH, and NHCO(C₁-C₆)alkyl;
R37, R38, R39, R40 are, independently of one another, H, or (C₁-C₆)alkyl; and the physiologically acceptable salts thereof.

3. The compound of either of claim 1 or 2, wherein
A is (C₃-C₇)alkyl, (C₀-C₂)alkylenearyl; a 5- to 10-membered mono- or bicyclic ring which may contain 0, 1, or 2 heteroatoms selected from the group consisting of N, O, and S, and the 5- to 10-membered ring may carry further substituents selected from the group consisting of F, Cl, Br, NO₂, CF₃, (C₁-C₆)alkyl, aryl, O—(C₁-C₆)alkyl, and NHCO(C₁-C₆)alkyl;
X is a bond, C(R8)(R9), O, or N(R12);
R8, R9, R12 are, independently of one another, H or (C₁-C₆)alkyl;
D is C(R41);
E is C(R42);
G is C(R43);
L is C(R44);
R1, R2, R3, R41, R42, R43, R44 are, independently of one another, H, F, Cl, CF₃, NO₂, O—(C₁-C₆)alkyl, (C₁-C₆)alkyl, O—(C₃-C₈)cycloalkyl, (C₀-C₂)alkylenearyl, —O—(C₀-C₃)alkylenearyl, N(R13)(R14), COO—(C₁-C₆)alkyl, CON(R15)(R16), N(R17)CO(R18), N(R19)SO₂(R20), or CO(R21);
R13, R14 are, independently of one another, H or (C₁-C₆)alkyl;
R15, R16 are, independently of one another, H or (C₁-C₆)alkyl;
R17, R 19 are, independently of one another, H or (C₁-C₆)alkyl;
R18, R20, R21 are, independently of one another, (C₁-C₆)alkyl, or aryl;
B is N(R24);
R24 is H or (C₁-C₆)alkyl;
R5 is H or (C₁-C₆)alkyl;
W is N;
T is C(R26);
R26 is H, (C₁-C₆)alkyl, or a bond to Y;
U is N(R27);
R27 is H, (C₁-C₆)alkyl, or a bond to Y;
Y is (C₁-C₃)alkylene, in which a carbon may be replaced by SO₂, C(R32)(R33) or CO;
R32, R33 are, independently of one another, H, (C₁-C₆)alkyl, or aryl;
R6, R7 are, independently of one another, H, (C₁-C₆)alkyl, (C₃-C₇)cycloalkyl, or R6 and Y or R6 and R⁷, together with the nitrogen atom to which they are bonded, form a 5- or 6-membered ring in which one or more carbons may be replaced by O or N, and the 5- or 6-membered ring may carry further substituents selected from the group consisting of (C₁-C₆)alkyl, aryl, CON(R37)(R38), N(R39)(R40), OH and NHCO(C₁-C₆)alkyl;
R37, R38, R39, R40 are, independently of one another, H or (C₁-C₆)alkyl; and the physiologically acceptable salts thereof.

4. A pharmaceutical composition comprising one or more of the compounds of claim 1 and a physiologically acceptable carrier.

5. (canceled)

6. A method for the prophylaxis or treatment of obesity, comprising administering to a mammal in need thereof an effective amount of the compound of claim 1, or a physiologically acceptable salt thereof.

7. A method for the prophylaxis or treatment of type II diabetes, comprising administering to a mammal in need thereof an effective amount of the compound of claim 1, or a physiologically acceptable salt thereof.

8-9. (canceled)

10. A method for preparing a pharmaceutical composition, comprising one or more of the compounds of claim 1, comprising mixing the active substance with a pharmaceutically suitable carrier and bringing said mixture into a form suitable for administration.

11. A method for the prophylaxis or treatment of arteriosclerosis or high blood pressures, comprising administering to a mammal in need thereof an effective amount of the compound of claim 1, or a physiologically acceptable salt thereof.

12. A method for normalizing lipid metabolism, comprising administering to a mammal in need thereof an effective amount of the compound of claim 1, or a physiologically acceptable salt thereof.

13. A method for the prophylaxis or treatment of paresthesia, depression, anxiety, anxiety neuroses, or schizophrenia, comprising administering to a mammal in need thereof an effective amount of the compound of claim 1, or a physiologically acceptable salt thereof.

14. A method for the prophylaxis or treatment of disorders associated with the circadian rhythms, comprising administering to a mammal in need thereof an effective amount of the compound of claim 1, or a physiologically acceptable salt thereof.

15. A method for the treatment of drug abuse, comprising administering to a mammal in need thereof an effective amount of the compound of claim 1, or a physiologically acceptable salt thereof.
